(19) United States
(12) Patent Application Publication
(10) Pub. No.: US 2025/0213711 A1
(43) Pub. Date: Jul. 3, 2025

(54) ANTI-CANAG ANTIBODY CONJUGATE

(71) Applicant: IKSUDA THERAPEUTICS LIMITED, Newcastle upon Tyne (GB)

(72) Inventors: Jenny Thirlway, Haxby, York (GB); Daniel John Williamson, Lupton (GB); Jutta Deckert, Lexington, MA (US); Justyna Mysliwy, Newcastle upon Tyne (GB); Robert Lutz, Wayland, MA (US)

(21) Appl. No.: 18/843,659

(22) PCT Filed: Mar. 6, 2023
(86) PCT No.: PCT/GB2023/050522

§ 371 Date: Sep. 3, 2024

(30) Foreign Application Priority Data
Mar. 4, 2022 (GB) 2203070.4

(51) Int. Cl. A61K 47/68; A61P 35/00; C07K 16/30

(52) U.S. Cl. CPC A61K 47/68035; A61K 47/6851; A61P 35/00; C07K 16/3092

(57) ABSTRACT

The present invention relates to antibody conjugates that target the CanAg antigen, and compositions (e.g., pharmaceutical compositions) comprising the antibody conjugates. Methods of using the antibody conjugates and compositions, including for the treatment of cancer, are also provided.

### FIELD OF THE INVENTION

The present invention relates to antibody conjugates that target the CanAg antigen, and compositions comprising the antibody conjugates. Methods of using the antibody conjugates and compositions, including for the treatment of cancer, are also provided.

### BACKGROUND OF THE INVENTION

Antibody-drug conjugate (ADC) technology is a target-oriented technology, which exploits the ability of an antibody to sensitively discriminate between healthy and diseased tissue to selectively deliver a cytotoxic payload. Three key elements define an ADC: the antibody, the cytotoxic drug (also called payload) and the linker connecting the drug to the antibody. ADCs are known for use as anticancer agents and function by using the antibody to target a specific antigen associated with cancerous cells and then releasing the drug payload under specific conditions to induce cell death. This enables the targeted delivery of a highly potent drug directly into the tumour, thereby reducing systemic exposure and toxicity to normal tissues. Accordingly, ADCs have significant potential to improve the treatment and survival of patients suffering from diseases such as cancer.

Despite the potential to use toxic payloads that are normally not tolerated by patients, a low therapeutic index (a ratio that compares toxic dose to efficacious dose) continues to be a problem and accounts for the discontinuance of many ADCs in clinical development. The selection of an appropriate target, antibody, cytotoxic payload, and the manner in which the antibody is linked to the payload have all been identified as key determinants of the safety and efficacy of ADCs. This indicates the level of complexity involved in developing ADCs that achieve the right combination of suitable target antigen, a stable linker, a potent cytotoxic payload, as well as an effective release technology.

The CanAg antigen has been suggested as one suitable target for selective antibody-based anticancer therapies based on its favourable expression pattern. CanAg is highly expressed in most pancreatic, biliary and colorectal cancers as well as in a significant proportion of gastric, uterine, non-small cell lung cancer, and bladder cancers. In contrast, only minimal expression of CanAg in normal tissue has been reported. Despite this, there are currently no anti-CanAg ADCs approved for use in the treatment of cancer. Cantuzumab mertansine and Cantuzumab ravtansine are two known ADCs that target CanAg, but neither compound has progressed further than Phase 2 clinical trials, possibly due to the limited efficacy observed against colorectal and pancreatic cancers.

Accordingly, there continues to be a need to identify and develop antibody-linker-drug combinations possessing effective efficacy, pharmacokinetics/pharmacodynamics, and a wide therapeutic index.

### SUMMARY OF INVENTION

In a first aspect, there is provided an antibody conjugate represented by Formula I or a pharmaceutically acceptable salt or solvate thereof:

Ab-(L-D)n Formula I:

wherein:
Ab is a humanised C242 antibody or antigen-binding fragment thereof;
D is a pyrrolobenzodiazepine dimer prodrug;
L is a linker connecting Ab to D;
n is an integer from 1 to 20;

Wherein:
D is represented by Formula (II):

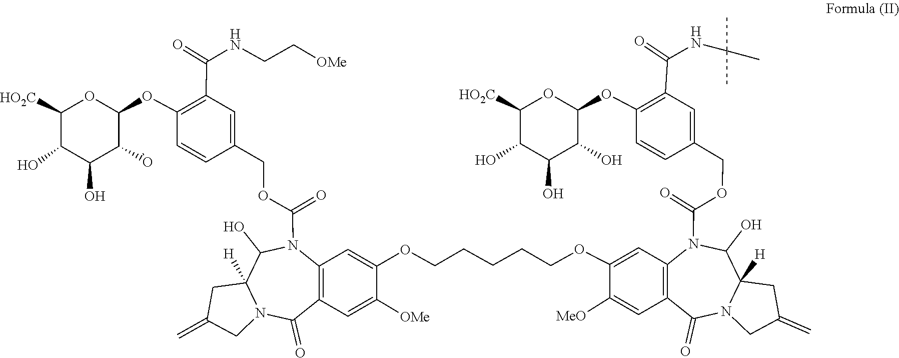

the linker comprises a central portion represented by Formula III, IV, V, VI or VII:

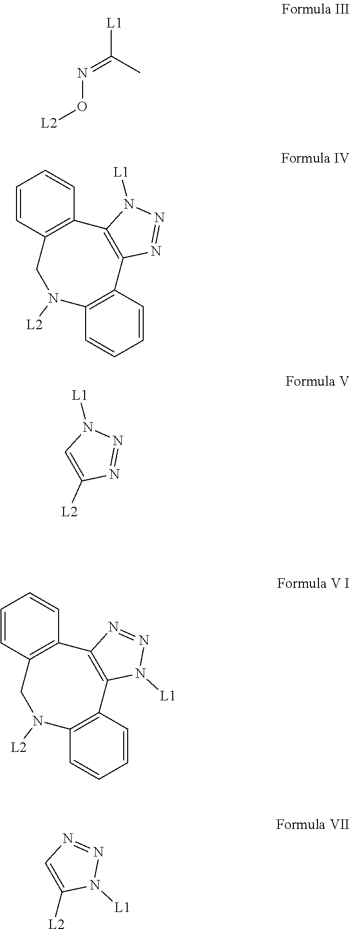

wherein:
L1 comprises a first connecting portion connecting the central portion to Ab; and
L2 comprises a second connecting portion connecting the central portion to D.

Alternatively, the first connecting portion L1 may connect the central portion to D and the second connecting portion L2 may connect the central portion to Ab.

The present inventors have found specific combinations of antibody, linkers and drug, which together achieve the desired characteristics. Advantageously, the specific ADCs of the present invention surprisingly exhibit improved safety and efficacy compared to known anti-CanAg ADCs.

In particular, a pyrrolobenzodiazepine (PBD) dimer cytotoxic payload is employed in the form of a prodrug according to Formula (II). PBDs are a known class of highly cytotoxic DNA cross-linking agents that exploit a different cellular target to the auristatin and maytansinoid tubulin inhibitor classes and a different mode of DNA damage to other DNA interacting payloads, such as calicheamicin. The prodrug form of the PBD dimer according to Formula (II) is more stable and exhibits lower cytotoxicity compared to conventional PBD drugs, which may suffer from poor stability in blood after administration. The prodrug is converted to an active form through cleavage of the glucuronic acid moieties by a β-glucuronidase enzyme, which is known to be upregulated in cancer cells relative to surrounding normal tissues (Fishman, W. H., J. Biol. Chem., 1947, 169 (2) p: 449). This may result in higher tumour selectivity of the active form of the drug and reduce the occurrence of side effects caused by premature decomposition of the linker by normal cells.

As alluded to above, linker stability is a critical factor in determining the efficacy and toxicity of the antibody-drug conjugate. Existing linkers, such as the widely-used maleimide attachment method, can suffer from non-specific release of payloads in non-tumorous tissues, leading to off-target toxicity and a limited therapeutic window. Advantageously, the linkers in accordance with the present invention, as described above, provide a stable connection between the antibody and the drug while allowing efficient cleavage of the drug in tumour cells.

The central portion of the linker connecting the antibody and the drug may comprise an O-substituted oxime according to Formula III. When the carbon atom of the oxime is substituted with a first connecting group that covalently links the oxime to the antibody, the oxygen atom of the oxime is substituted with a second connecting group that covalently links the oxime to the drug (D). Alternatively, when the carbon atom of the oxime is substituted with a connecting group that covalently links the oxime to the drug, the oxygen atom of the oxime is substituted with a connecting group that covalently links the oxime to the antibody.

In other embodiments, the central portion of the linker may comprise a substituted triazole according to formula IV, V, VI or VII, instead of an oxime. Advantageously, triazoles can be formed by click chemistry reactions carried out under mild conditions, which can be performed in the presence of an antibody without denaturing occurring. Further, an azide-alkyne click chemistry reaction, for example, may produce a triazole in a high yield and with high reaction specificity. Therefore, even though antibodies have various functional groups (for example, amines, carboxyls, carboxamides, and guanidiniums), a click chemistry reaction may be performed, for example, without affecting the amino acid side chains of the antibody. As would be appreciated by those skilled in the art, formulae VI and VII are regioisomers of formulae IV and V, respectively.

Any humanised antibody or an antigen-binding fragment thereof that can target CanAg may be used in accordance with the invention. Suitably, the antibody is a humanised C242 antibody or an antigen binding-fragment thereof. The humanized C242 (HuC242 or Cantuzumab) binds to the CA242 epitope on the extracellular domain of the CanAg antigen. Examples of humanised C242 for use in the present invention may comprise one or more amino acid sequences from SEQ ID NO: 5-10 or 13-18.

Suitably, the linker is covalently bound to the antibody by a thioether bond. For example, the thioether bond may comprise a sulfur atom of a cysteine of the antibody. Cysteine-based conjugation methods offer greater control of drug loading, i.e. the drug-to-antibody ratio (DAR) and homogeneity, compared to lysine conjugation methods. Greater ADC homogeneity is known to be associated with improved pharmacokinetics and efficacy and reduced off-target toxicity. The covalent thioether bond may be formed using existing thiol groups or by introducing thiol groups in a precursor step, for example by reacting one or more functional groups of the antibody to produce a thiol group, or by introducing a thiol group or a precursor thereof into the antibody. By way of example, this may involve the step of introducing a cysteine residue into the antibody at a site where it is desired to bind the linker to the antibody. This may be useful in situations where a convenient cysteine residue for reaction according to the present invention is not present in a starting or wild-type antibody. Conveniently, this may be achieved using site directed mutagenesis of the antibody, the use of which is well established in the art.

The first connecting portion may include at least one isoprenyl unit represented by Formula VIII:

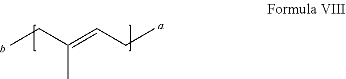

When the first connecting portion includes at least one isoprenyl unit represented by Formula VIII, the carbon atom (a) of the isoprenyl unit forms a thioether bond with a sulfur atom, preferably of a cysteine, of the antibody, thereby covalently linking the isoprenyl group and the antibody. The carbon atom (b) of the isoprenyl group covalently links the isoprenyl group to the central portion of the linker.

Advantageously, antibody prenylation of a C-terminal amino acid sequence to install a modified isoprenyl unit has been described that allows for attachment of a drug or other active agent to the antibody in a mild and site-specific manner. This allows for the preparation of homogeneous ADCs having a defined number of drugs, which is known to improve pharmacokinetics and efficacy and is more desirable from a regulatory perspective.

The antibody may comprise an amino acid motif, preferably at a C-terminus of the antibody, that is recognized by an isoprenoid transferase. The amino acid motif may be a sequence selected from CXX, CXC, XCXC, XXCC, and CYYX, wherein C represents cysteine; Y, independently for each occurrence, represents an aliphatic amino acid; and X, independently for each occurrence, represents glutamine, glutamate, serine, cysteine, methionine, alanine, or leucine. Suitably, the thioether bond may comprise a sulfur atom of a cysteine of the amino acid motif.

Optionally, the amino acid motif may be a sequence CYYX, and Y, independently for each occurrence, represents alanine, isoleucine, leucine, methionine, or valine. For example, the amino acid motif may be CVIM or CVLL. At least one of the seven amino acids preceding the amino acid motif may be glycine. For example, at least three of the seven amino acids preceding the amino acid motif are each independently selected from glycine and proline. Suitably, each of the one, two, three, four, five, six, seven, eight, nine, or ten amino acids preceding the amino acid motif is glycine, preferably seven. Optionally, the antibody comprises the amino acid sequence GGGGGGGCVIM, preferably at a C-terminus.

Alternatively, the first connecting portion is represented by Formula IX:

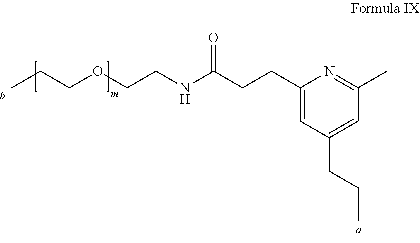

Wherein:
^{a} denotes a point of attachment to Ab;
^{b} denotes a point of attachment to the central portion; and
m is an integer from 1 to 10.

Advantageously, vinylpyridine-based linkers in accordance with Formula IX have been shown to react selectively and irreversibly with thiol groups on an antibody to form highly stable thioether bonds. As noted above, linker stability is critical to the efficacy and toxicity of ADCs.

Suitably, the second connecting portion may comprise at least one polyethylene glycol unit represented by Formula X:

—(CH₂CH₂O)ₒ Formula X

wherein o is an integer from 1 to 10.

Alternatively, the second connecting portion may be represented by Formula XI:

—(CH₂)ₚ—NHC(O)—(CH₂CH₂)O)_{q}(CH₂)ᵣ—C(O)—NH—(CH₂)ₛ—C(O) Formula XI

wherein:
p is an integer from 1 to 10;
q is an integer from 1 to 20;
r is an integer from 1 to 10; and
s is an integer from 1 to 10.

Suitably, the antibody conjugate may comprise a structure selected from:

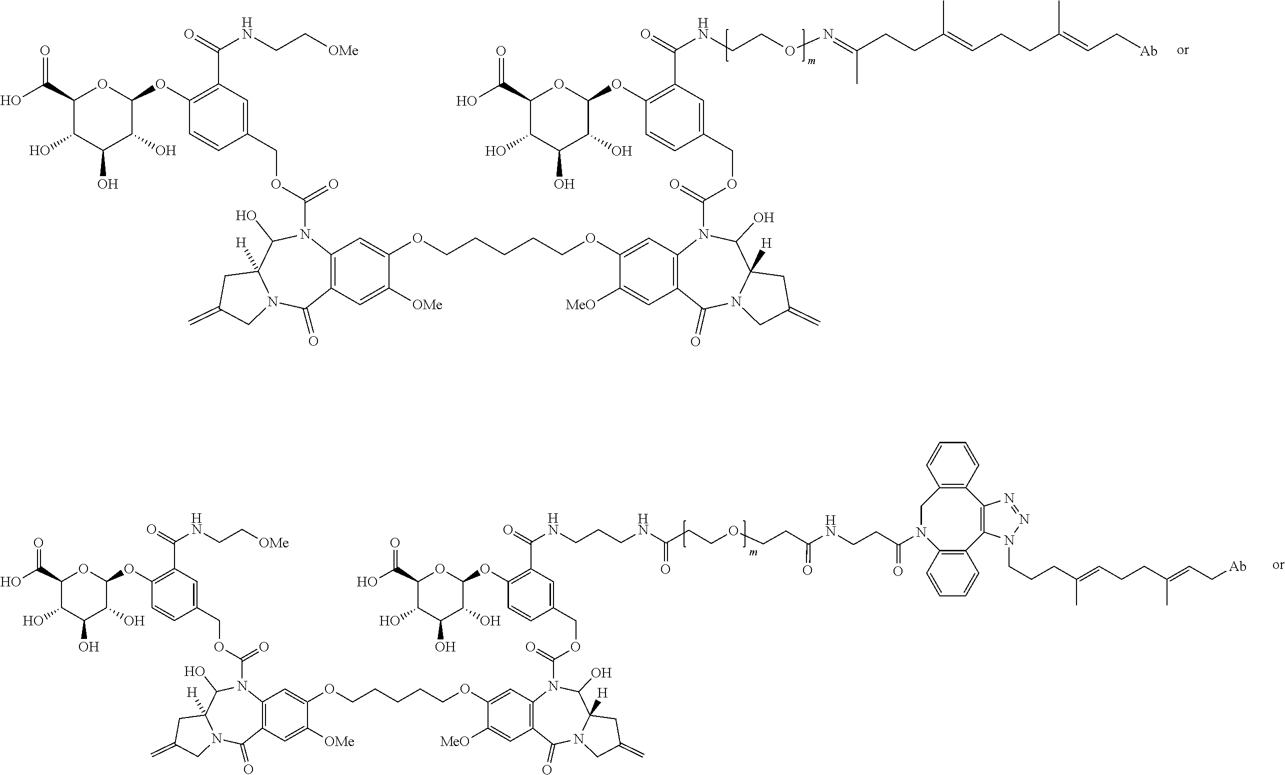

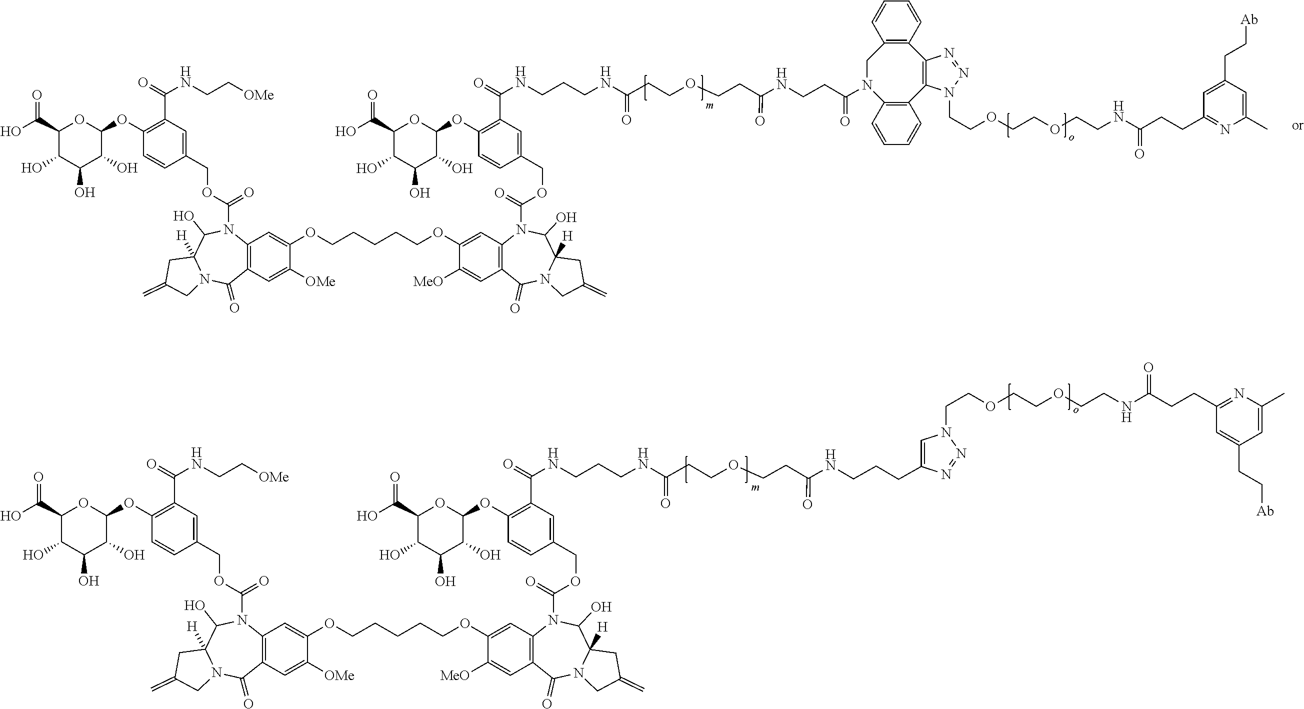

Wherein:
m is an integer from 0 to 20; and
is an integer from 0 to 10.

In a second aspect, there is provided a pharmaceutical composition comprising an antibody conjugate according to the first aspect; and one or more pharmaceutically acceptable excipients, diluents, or carriers.

Suitably, the pharmaceutical composition may be for use as a medicament. For example, the medicament may be for use in the treatment of cancer. Optionally, the cancer may be selected from the group consisting of lung cancer, small cell lung cancer, gastrointestinal cancer, colorectal cancer, bladder cancer, pancreatic cancer, biliary cancer, cervical cancer and uterine cancer. For example, the cancer may be pancreatic cancer.

In another aspect, there is provided use of the pharmaceutical composition according to the second aspect in the preparation of a medicament for the treatment of cancer.

In another aspect, there is provided a method of treating cancer in a subject in need thereof, comprising the step of administering a therapeutic amount of the pharmaceutical composition according to the second aspect to the subject.

Optionally, the cancer may be selected from the group consisting of lung cancer, small cell lung cancer, gastrointestinal cancer, colorectal cancer, bladder cancer, pancreatic cancer, biliary cancer, cervical cancer, and uterine cancer. For example, the cancer may be pancreatic cancer.

Embodiments of the present invention will now be described by way of example and not limitation with reference to the accompanying figures.

### BRIEF DESCRIPTION OF FIGURES

The accompanying drawings illustrate presently exemplary embodiments of the disclosure, and together with the general description given above and the detailed description of the embodiments given below, serve to explain, by way of example, the principles of the disclosure.

FIG. 1 shows binding of humanised antibodies to CanAg positive Colo205 cells;

FIG. 2 shows a PLRP chromatogram (A214 nm) of an ADC-1 with a DAR of 2.3. Numbers designate the amount of drug conjugated to light (L) or heavy (H) chain;

FIG. 3 shows a SEC chromatogram (A214 nm) of an ADC-1 with a DAR of 2.3;

FIG. 4 shows a HIC chromatogram (A214 nm) of ADC-2 with a DAR of 2 after conjugation of compound 2 to the prenylated HC1+LC1 intermediate and purification by semi-preparative HIC;

FIG. 5 shows a SEC chromatogram (A214 nm) of an ADC-2 with a DAR of 2 after purification;

FIG. 6 shows a HIC chromatogram (A214 nm) of an ADC-3 with a DAR of 2 after conjugation of compound 5 to the prenylated HC1LC1 intermediate and purification by semi-preparative HIC;

FIG. 7 shows a SEC chromatogram (A214 nm) of an ADC-3 with a DAR of 2 after purification;

FIG. 8 shows SEC analysis of Cantuzumab ravtansine at 280 nm;

FIG. 9 shows CanAg expression level on SNU-16, Colo-205, HT29, BxPC3 and NCI-N87 cells;

FIG. 10 shows β-glucuronidase activity in Colo-205, HT29, BxPC3 and NCI-N87 cells;

FIG. 11 shows the in vitro activity of ADC-2 and ADC non-binding control on Colo205 cells;

FIG. 12 shows the in vitro activity of ADC-2 and ADC non-binding control on BxPC-3 cells;

FIG. 13 shows the in vitro activity of ADC-2 and ADC non-binding control on HT29 cells;

FIG. 14 shows the in vitro activity of ADC-2 and ADC non-binding control on N87 cells;

FIG. 15 shows the in vitro activity of ADC-1, ADC-2 and ADC-3 on Colo-205 cells;

FIG. 16 shows the in vitro activity of ADC-3, Cantuzumab ravtansine and ADC non-binding control on Colo-205 cells;

FIG. 17 shows the in vivo activity of ADC-2 and ADC-3 in Colo-205 xenograft (A), with body weight changes shown in (B);

FIG. 18 shows the in vivo activity of ADC-1, ADC-2 and ADC-3 in Colo-205 xenograft (A), with body weight changes shown in (B);

FIG. 19 shows the in vivo activity of Cantuzumab ravtansine in Colo-205 xenograft (A), with body weight changes shown in (B);

FIG. 20 shows the in vivo activity of ADC-2, ADC-3 and Cantuzumab ravtansine in BxPC-3 xenograft (A), with body weight changes shown in (B);

FIG. 21 shows the in vivo activity of Cantuzumab ravtansine in BxPC-3 xenograft (A), with body weight changes shown in (B);

FIG. 22 shows the in vivo activity of ADC-3 and non-binding ADC control in NCI-N87 xenograft (A), with body weight changes shown in (B);

FIG. 23 shows the in vivo activity of Cantuzumab ravtansine (A) and Enhertu (B) ADCs in NCI-N87 xenograft, with body weight changes shown in (C) and (D), respectively; and

FIG. 24 shows the alignment of the heavy chain (HC) and light chain (LC) sequences for antibody humanisation. CDR regions are shown underlined. Amino acids belonging to the signal peptide are shown in bold font.

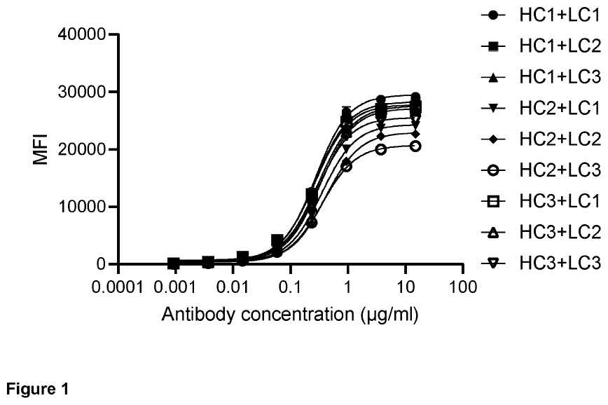

### DETAILED DESCRIPTION

The present invention relates to antibody conjugates that target the CanAg antigen, and compositions (e.g., pharmaceutical compositions) comprising the antibody conjugates. Methods of using the antibody conjugates and compositions, including for the treatment of cancer, are also provided.

The term “antibody” means an immunoglobulin molecule that recognises and specifically binds to a target, such as a protein, polypeptide, peptide, carbohydrate, polynucleotide, lipid, or combinations of the foregoing through at least one antigen recognition site within the variable region of the immunoglobulin molecule. References to antibodies include immunoglobulins whether natural or partly or wholly synthetically produced. The term also covers any polypeptide or protein comprising an antigen binding domain. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations.

The term “antibody fragment” refers to a portion of an intact antibody and refers to the antigenic determining variable regions of an intact antibody. Examples of antibody fragments include, but are not limited to Fab, Fab′, F(ab′)2, and Fv fragments, linear antibodies, nanobodies, single chain antibodies, bispecific and multispecific antibodies formed from antibody fragments.

“Prodrug” refers to a compound that is metabolised, for example hydrolysed, in the host after administration to form a biologically active molecule. Typical examples of prodrugs include compounds that have biologically labile or cleavable protecting groups on a functional moiety of the active compound.

The terms “cancer” and “cancerous” refer to or describe the physiological condition in mammals in which a population of cells are characterised by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, fallopian tube cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancers.

“Tumour” refers to any mass of tissue that result from excessive cell growth or proliferation, either benign (noncancerous) or malignant (cancerous) including precancerous lesions.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The term “pharmaceutical composition” refers to a preparation which is in such form as to permit the biological activity of the active ingredient to be effective, and which contains no additional components which are unacceptably toxic to a subject to which the formulation would be administered. Such formulation can be sterile.

An “effective amount” as disclosed herein is an amount sufficient to carry out a specifically stated purpose. An “effective amount” can be determined empirically and in a routine manner, in relation to the stated purpose.

The term “therapeutically effective amount” refers to an amount of an ADC or other drug effective to “treat” a disease or disorder in a subject or mammal. In the case of cancer, the therapeutically effective amount of the drug can reduce the number of cancer cells; reduce the tumour size; inhibit (i.e., slow to some extent and in a certain embodiment, stop) cancer cell infiltration into peripheral organs; inhibit (i.e., slow to some extent and in a certain embodiment, stop) tumour metastasis; inhibit, to some extent, tumour growth; and/or relieve to some extent one or more of the symptoms associated with the cancer. See the definition of “treating” below. To the extent the drug can prevent growth and/or kill existing cancer cells, it can be cytostatic and/or cytotoxic. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

Terms such as “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent and/or slow the development of a targeted pathologic condition or disorder. Thus, those in need of treatment include those already with the disorder; those prone to have the disorder; and those in whom the disorder is to be prevented. In certain embodiments, a subject is successfully “treated” for cancer according to the methods of the present invention if the patient shows one or more of the following: a reduction in the number of or complete absence of cancer cells; a reduction in the tumour size; inhibition of or an absence of cancer cell infiltration into peripheral organs including, for example, the spread of cancer into soft tissue and bone; inhibition of or an absence of tumour metastasis; inhibition or an absence of tumour growth; relief of one or more symptoms associated with the specific cancer; reduced morbidity and mortality; improvement in quality of life; reduction in tumourigenicity, tumourigenic frequency, or tumourigenic capacity, of a tumour; reduction in the number or frequency of cancer stem cells in a tumour; differentiation of tumourigenic cells to a non-tumourigenic state; or some combination of effects.

The disclosure provides antibody-drug conjugates of antibodies that bind to the CanAg antigen. CanAg is strongly expressed in most pancreatic, biliary, and colorectal cancers. It is also expressed in a substantial proportion of gastric cancers, uterine cancers, non-small cell lung cancers, and bladder cancers. In contrast, only minimal expression of CanAg in normal tissue has been reported. As such, CanAg appears to be a suitable candidate for mAb-based anticancer therapies. However, there are currently no marketed ADCs that target CanAg. Cantuzumab mertansine and cantuzumab ravtansine are two known ADCs that target CanAg, but neither compound progressed further than Phase 2 clinical trials. The present invention has surprisingly found that particular ADCs as claimed provide improved ADCs which target CanAg.

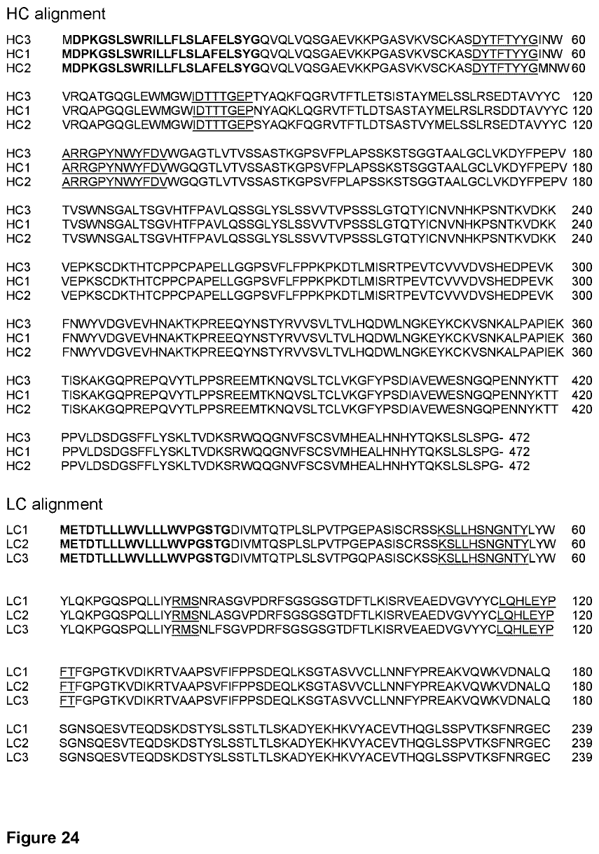

Any humanized antibody or an antigen-binding fragment thereof that can target CanAg may be used in accordance with the invention. In order to maintain binding affinity, the humanisation process may comprise identifying CDR regions and residues interacting with CDRs or in VH-VL interfaces and preserving these regions in the humanised antibody. Suitably, the antibody in accordance with the invention is a humanised antibody in which the CDRs underlined in FIG. 24 are preserved, or an antigen binding-fragment including these regions. Examples of humanised C242 for use in the present invention may comprise one or more amino acid sequences from SEQ ID NO: 5-10 or 13-18.

The antibody conjugates according to the present invention may be useful in a variety of applications including, but not limited to, therapeutic treatment methods, such as the treatment of cancer. In certain embodiments, the agents are useful for inhibiting tumour growth, inducing differentiation, reducing tumour volume, and/or reducing the tumourigenicity of a tumour. The methods of use may be in vitro, ex vivo, or in vivo methods. In certain embodiments, the disease treated with the antibody conjugate or compositions comprising the antibody conjugate is a cancer. In certain embodiments, the cancer is characterised by tumours expressing CanAg.

The present invention provides for methods of treating cancer comprising administering a therapeutically effective amount of the antibody conjugates or compositions thereof to a subject (e.g., a subject in need of treatment). In certain embodiments, the cancer is a cancer selected from the group consisting of lung cancer, small cell lung cancer, gastrointestinal cancer, colorectal cancer, bladder cancer, pancreatic cancer, biliary cancer, cervical cancer and uterine cancer. In certain embodiments, the cancer is pancreatic cancer. In certain embodiments, the cancer is colorectal cancer. In certain embodiments, the subject is a human.

The pharmaceutical compositions of the present invention can be administered in any number of ways for either local or systemic treatment. Administration can be pulmonary (e.g., by inhalation or insufflation of powders or aerosols, including by nebulizer; intratracheal, intranasal, epidermal and transdermal); oral; or parenteral including intravenous, intraarterial, subcutaneous, intraperitoneal or intramuscular injection or infusion; or intracranial (e.g., intrathecal or intraventricular) administration.

For the treatment of the disease, the appropriate dosage of an antibody or agent of the present invention depends on the type of disease to be treated, the severity and course of the disease, the responsiveness of the disease, whether the antibody conjugate administered for therapeutic or preventative purposes, previous therapy, patient's clinical history, and so on all at the discretion of the treating physician. The antibody conjugate or compositions thereof can be administered one time or over a series of treatments lasting from several days to several months, or until a cure is effected or a diminution of the disease state is achieved (e.g. reduction in tumour size). Optimal dosing schedules can be calculated from measurements of drug accumulation in the body of the patient and will vary depending on the relative potency of an individual antibody or agent. The administering physician can easily determine optimum dosages, dosing methodologies and repetition rates.

### Experimental Data and Discussion

The invention is further illustrated by the following examples, which are not to be construed in any way as imposing limitations to the scope of this invention as defined by the appended claims.

### Example 1. Development of Humanised Anti-CanAg Antibodies

The humanisation design of the parental antibody was performed using in silico analysis. A 3D structure of the parental antibody using homology modeling was generated. Acceptor frameworks were identified based on the overall sequence identity across the framework, matching interface position, and similarly classed CDR canonical positions. Three heavy chain (HC) frameworks and three light chain (LC) frameworks were selected for the humanisation design.

Humanised antibodies were designed by creating multiple hybrid sequences that fuse select parts of the parental antibody sequence with the human framework sequences. Using the 3D structure, these humanized sequences were methodically analysed by eye and computer modelling to isolate the sequences that would most likely retain antigen binding (focusing on key residues supporting CDR loop and VHVL interface). The goal was to maximise the amount of human sequence in the final humanised antibodies while retaining the original antibody specificity.

Three humanised VH and three humanised VL sequences were designed: “HV1-18 BM (HC1),” “HV1-46 BM (HC2),” and “HV1-8 BM (HC3)”; “KV2-40 BM (LC1) and “KV2-28 BM (LC2),” and “KV2D-29 BM (LC3)”. The humanness score (T20 score) for the humanised antibodies was calculated by analysing the primary sequences of the variable regions using the method described in Gao et al (Monoclonal antibody humanness score and its applications; BMC Biotechnology, 13:55, 2013).

The humanised heavy and light chains were then combined to create variant fully humanised antibodies. Nine (9) combinations of humanised heavy and light chains were tested for their expression level and antigen-binding affinity to identify antibodies that perform similarly to the chimeric parental antibody. The antibodies tested are shown in Table 1 below. A 0.01 L transient production (TunaCHO™ 7-day) for the nine humanised variants and chimeric parental antibody was performed. All clones were purified by Protein A (see Table 1 for production yield).

TABLE 1
Production yield of the antibodies in mg/L.

| Antibody name | Yield mg/L |
|---|---|
| HC1 + LC1 | 35 |
| HC1 + LC2 | 31 |
| HC1 + LC3 | 39 |
| HC2 + LC1 | 36 |
| HC2 + LC2 | 37 |
| HC2 + LC3 | 33 |
| HC3 + LC1 | 36 |
| HC3 + LC2 | 36 |
| HC3 + LC3 | 33 |

The affinity of the nine humanised antibody combinations for CanAg positive Colo205 cells was evaluated by flow cytometry as follows. 20×10⁵ Colo205 cells were aliquoted per well and washed once with FACS buffer (2% FBS in PBS). Humanised antibodies and isotype control antibodies (Anti-HEL-Human-IgG1 (N297A), catalog: B109801, brand: BIOINTRON) were diluted with FACS buffer diluted (8 concentrations starting at 100 μg/ml with 2-fold dilutions), then 100 μL was added to each well with cells. Cells were incubated at 4° C. for 60 minutes followed by washing with FACS buffer twice. Secondary antibody AF488 Goat anti-human IgG (H+L) (catalog: A11013, brand: Invitrogen) was diluted 1:1000 with FACS buffer, then 100 uL added to each well and incubated at 4° C. for 30 minutes. After the incubation, cells were re-suspended in 200 uL FACS buffer for flow cytometry analysis. MFI (mean fluorescence intensity) was used to calculate EC50. The EC50 values for binding of humanised antibodies to Colo205 cells determined by FACS are shown in Table 2.

TABLE 2
EC50 values for binding of humanised antibodies
to Colo205 cells determined by FACS.

| | Binding EC50 (μg/ml) |
|---|---|
| Clone | MFI curve |
| HC1 + LC1 | 0.2737 |
| HC1 + LC2 | 0.2676 |
| HC1 + LC3 | 0.3048 |
| HC2 + LC1 | 0.3506 |
| HC2 + LC2 | 0.4078 |
| HC2 + LC3 | 0.3540 |
| HC3 + LC1 | 0.2664 |
| HC3 + LC2 | 0.3269 |
| HC3 + LC3 | 0.2940 |

As can be seen from Table 2, all humanised antibodies tested (HC1+LC1, HC1+LC2, HC1+LC3, HC2+LC1, HC2+LC2, HC2+LC3, HC3+LC1, HC3+LC2 and HC3+LC3) exhibited comparable affinity and expression yield in TunaCHO™ cell transient expression system.

### Example 2. ADC-1

ADC-1 was produced by vinylpyridine-mediated cysteine modification using compound 1 and compound 2 below.

### Synthesis of Compound 1

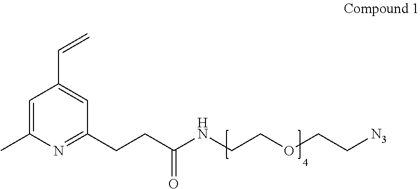

Lithium 3-(6-methyl-4-vinylpyridin-2-yl) propanoate (17.5 g, 87.6 mmol) and 2-(2-(2-azidoethoxy) ethoxy) ethan-1-amine (23.0 g, 87.6 mmol, commercially available from BroadPharm, catalogue number: BP-21615) were dissolved in dimethylformamide (525.0 mL). N-(3-Dimethylaminopropyl)-N′-ethylcarbodiimide hydrochloride (33.6 g, 175 mmol) was added portion-wise at 0° C. Diisopropylethylamine (61.0 mL, 350.0 mmol) was added dropwise and the mixture stirred for 12 hours. The reaction was quenched by addition of aqueous LiCl (1.0 L, 5.0% wt/vol) and washed with ethyl acetate (3×500 mL). The organic extracts were combined, dried over MgSO₄ and evaporated to dryness to afford crude N-(2-(2-(2-azidoethoxy) ethoxy)ethyl)-3-(6-methyl-4-vinylpyridin-2-yl) propenamide (Compound 1). The crude product was purified by column chromatography (Si₂O, dichloromethane:methanol gradient 98:2-96:4) to afford Compound 1 as an orange oil (21.5 g, 56% yield). 1H NMR (CDCl₃, 400 MHZ) δ ppm: 6.97 (2H, d) 6.53 (1H, t), 5.90 (1H, d), 5.42 (1H, d), 3.63 (14H, m), 3.44 (2H, d), 3.40 (4H, m), 3.04 (2H, t), 2.62 (2H, t), 2.45 (3H, s). LCMS (ESI+): Compound 1 (C₂₁H₃₃N₅O₅) Theoretical: 436.25 [M+1]¹⁺. Found: 436.28 [M+1]¹⁺.

Compound 2, shown below, was synthesised by the method described in WO2018182341, which is hereby incorporated by reference in its entirety.

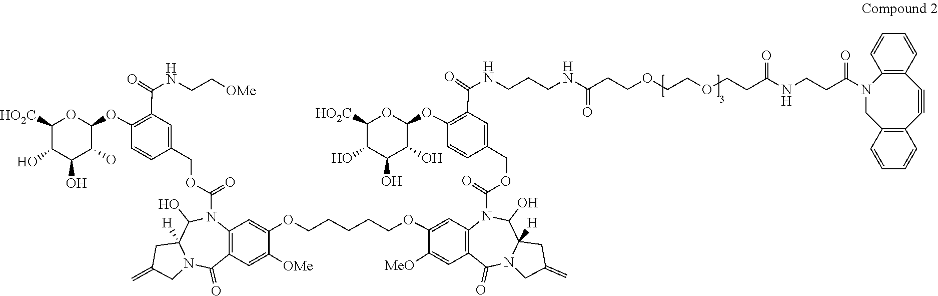

### ADC-1 Generation

HC1+LC1 antibody was partially reduced with 2.8 molar equivalents of TCEP and conjugated to compound 1 in a 4-fold molar excess and in the presence of 1.2% (v/v) dimethylacetamide (DMA), at pH 7.4 for ≥18 hours at 25° C. After isolation by desalting, the resulting intermediate was conjugated to compound 2 in a 2.5-fold molar excess and in the presence of 0.7% (v/v) dimethylacetamide (DMA), at pH 7.4 for ≥4 hours at 25° C. The conjugate was purified by desalting column to remove excess free drug and solvent and re-buffered to phosphate-buffered saline (PBS), PH 7.4.

### Determination of Drug Antibody Ratio (DAR) for ADC-1

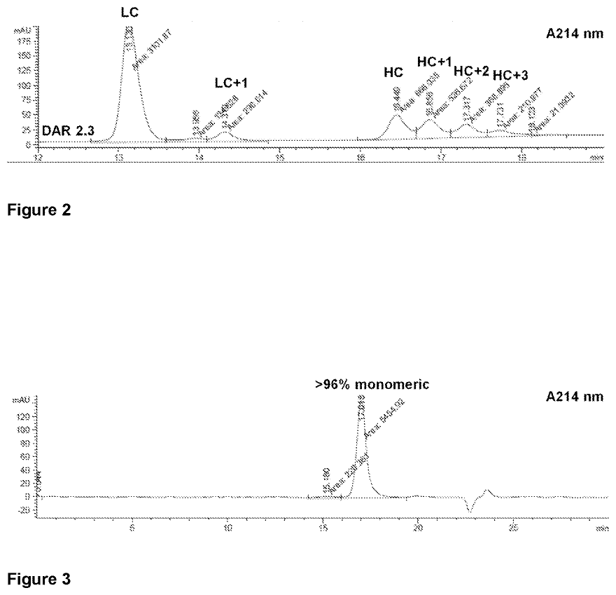

Determination of average drug-load and drug-load distribution is crucial for ADC generation, as these factors effect the potency and pharmacokinetics of the ADC. DAR determination of ADC-1 was accomplished by Polymer-Linked Reverse-Phase (PLRP) chromatography with an Agilent PLRP-S (1000 A, 2.1×50 mm, 5 μm) column. Separation of dithiothreitol (DTT) reduced conjugate via a PLRP column afforded well resolved peaks corresponding to unconjugated or drug conjugated antibody light and heavy chains, as shown in FIG. 2. The DAR value was determined to be 2.3 for ADC-1. Peak separation was performed using the following procedure; Buffer A (H₂O+0.1% TFA) and Buffer B (MeCN+0.1% TFA); Gradient; 0-3 min 25% buffer B; 3-28 min=25-50% buffer B; 28-31 min=95% buffer B; 31-40 min=25% buffer B.

### Determination of Monomeric Content of ADC-1

Size-exclusion chromatography (SEC) was employed to determine the degree to which the conjugate had aggregated during conjugation using a MAbPac™ SEC-1 column (5 μM, 300 Å, 7.8×300 mm). Elution was performed in 20 mM MES sodium salt, 150 mM NaCl, 5% MeCN, pH 6.0. ADC-1 shows more than 96% monomeric content, as shown in FIG. 3.

### Example 3. ADC-2

ADC-2 was prepared using protein prenylation of a C-terminal amino acid sequence to install a modified isoprenoid unit that allows for attachment of the drug to the antibody in a mild and site-specific manner. Such prenylation of an antibody is described, for example, in U.S. Patent Publication No. 2012/0308584, U.S. Pat. No. 9,919,057, PCT Publication No. WO 2017/089890 and PCT Publication No. WO 2017/089895, the contents of which are fully incorporated by reference herein.

In this example, the antibody was modified with an isoprenoid derivative functionalised with an azide group, shown below as Compound 3, for coupling with Compound 2 via click chemistry using the procedure described below. Compound 3 was prepared by the method described in US2012/0308584.

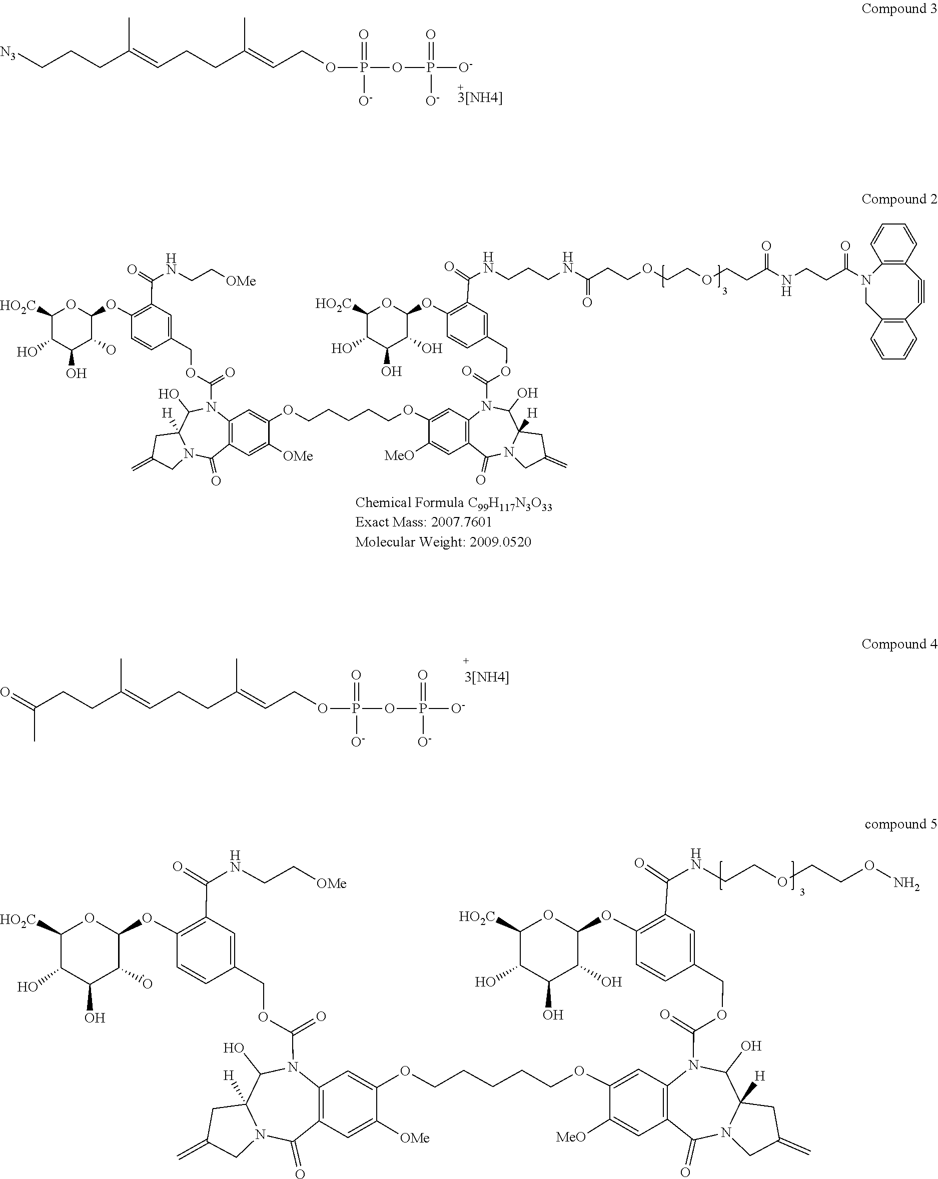

### ADC-2 Generation

HC1LC1 antibody with CAAX tag was prenylated with 8.3 molar equivalents of compound 3 in the presence of 0.2 μM FTase, 100 UM DTT, 500 mM Tris-HCl, 0.1 mM ZnCl₂ and 50 mM MgCl₂ at pH 7.4 for 4 hours at 30° C. The resulting intermediate was desalted into PBS, pH 7.4. Prenylated intermediate was conjugated with 2.5 molar equivalents of compound 2 in the presence 0.45% DMA for 2 hours at 30° C. ADC-2 was purified by semi-preparative HIC using a Phenyl phase HIC column (Tosoh Bioscience, L×I.D 7.5 cm×7.5 mm) using the following procedure; Buffer A (50 mM potassium phosphate+0.5 M ammonium sulphate, pH 7.0) and Buffer B (50 mM potassium phosphate+30% (v/v) MeCN, pH 7.0); Gradient—0-30 mins=0-100% buffer B; 30-32 min=100% buffer B; 32-32.1 min=100-0% buffer B; 32.1-49 min=0% buffer B; Flow rate-0.8 mL/min. Sample was desalted to phosphate buffered saline (PBS), pH 7.4.

### Determination of Drug Antibody Ratio (DAR) for ADC-2

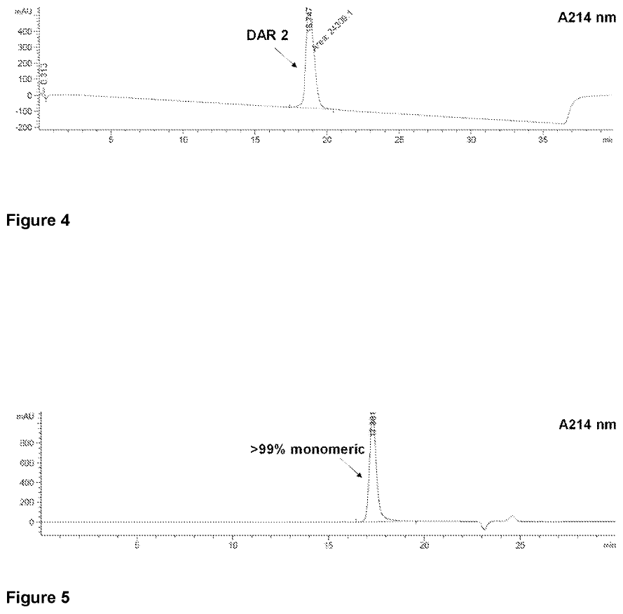

DAR determination for the ADC-2 was accomplished by Hydrophobic Interaction chromatography (HIC) with a Tosoh Biosciences HIC column (phase Butyl, L×I.D 3.5 cm 4 4.6 mm, 2.5 μM particle size). Separation of conjugate sample via a HIC column afforded one peak corresponding to the antibody conjugated to two drugs, as shown in FIG. 4. Elution was performed using the following procedure: Buffer A (25 mM sodium phosphate, 1.5 M ammonium sulphate, pH 7.0) and buffer B (25 mM sodium phosphate, 25% isopropanol pH 7.0); Gradient—0-30 min=0-100% buffer B; 30-35 min=0% buffer B.

### Determination of Monomeric Content of ADC-2

Size-exclusion chromatography (SEC) was employed to determine the degree to which the conjugate had aggregated during conjugation using a MAbPac™ SEC-1 column (5 μM, 300 Å, 7.8×300 mm). Elution was performed in 20 mM MES sodium salt, 150 mM NaCl, 5% MeCN, pH 6.0. ADC-2 shows more than 99% monomeric content, as shown in FIG. 5.

### Example 4. ADC-3

In this example, the antibody was modified with an isoprenoid derivative functionalised with a ketone group, shown above as Compound 4, for coupling with Compound 5 via oxime-forming chemistry using the procedure described below. Compound 4 was prepared by the method described in US2012/0308584. Compound 5 was synthesised by the method described in WO2018182341.

### ADC-3 Generation

HC1LC1 antibody with CAAX tag was prenylated with 6.25 molar equivalents of compound 4 in the presence of 0.2 μM FTase, 250 μM DTT, 500 mM Tris-HCl, 0.1 mM ZnCl₂ and 50 mM MgCl₂ at pH 7 for >18 hours at 30° C. The resulting intermediate was desalted into PBS, PH 7.4. Prenylated intermediate was conjugated with 10 molar equivalents of compound 5 in the presence 10% DMSO for 6 hours at 30° C. ADC-3 was purified by semi-preparative HIC using a Phenyl phase HIC column (Tosoh Bioscience, L×I.D 7.5 cm×7.5 mm) using the following procedure; Buffer A (50 mM potassium phosphate+0.5 M ammonium sulphate, pH 7.0) and Buffer B (50 mM potassium phosphate+30% (v/v) MeCN, pH 7.0); Gradient—0-30 mins=0-100% buffer B; 30-32 min=100% buffer B; 32-32.1 min=100-0% buffer B; 32.1-49 min=0% buffer B; Flow rate-0.8 mL/min. Sample was desalted to phosphate buffered saline (PBS), pH 7.4.

### Determination of Drug Antibody Ratio (DAR) for ADC-3

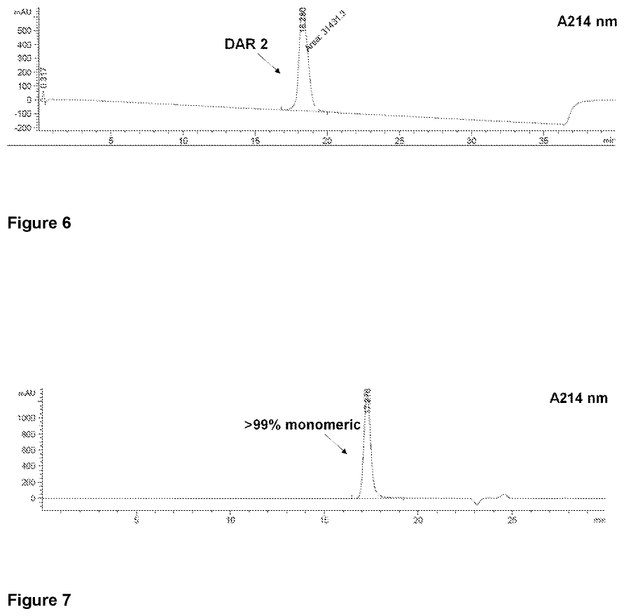

DAR determination for the ADC-3 was accomplished by Hydrophobic Interaction chromatography (HIC) with a Tosoh Biosciences HIC column (phase Butyl, L×I.D 3.5 cm 4 4.6 mm, 2.5 μM particle size). Separation of the conjugate sample via a HIC column afforded one peak corresponding to the antibody conjugated to two drugs, as shown in FIG. 6. Elution was performed using the following procedure: Buffer A (25 mM sodium phosphate, 1.5 M ammonium sulphate, pH 7.0) and buffer B (25 mM sodium phosphate, 25% isopropanol pH 7.0); Gradient—0-30 min=0-100% buffer B; 30-35 min=0% buffer B. Sample was desalted to phosphate buffered saline (PBS), pH 7.4.

### Determination of Monomeric Content of ADC-3

Size-exclusion chromatography (SEC) was employed to determine the degree to which the conjugate had aggregated during conjugation using a MAbPac™ SEC-1 column (5 μM, 300 Å, 7.8×300 mm). Isocratic elution was performed in 20 mM MES sodium salt, 150 mM NaCl, 5% MeCN, pH 6.0. ADC-3 shows more than 99% monomeric content, as shown in FIG. 7.

### Example 5. Cantuzumab Ravtansine Comparator

As described above, cantuzumab ravtansine is a humanised antibody-drug conjugate targeting CanAg. Cantuzumab ravtansine comprises cantuzumab conjugated to the cytotoxic maytansinoid drug, ravtansine, shown as Compound 6 below.

### Preparation of Cantuzumab Ravtansine

HuC242 (Cantuzumab antibody) at 5.35 mg/mL in PBS buffer was preconditioned for conjugation by the addition of 5% v/v 0.5 M borate 25 mM EDTA to achieve pH 8.2. 6 equivalents of compound 6 over antibody were added as a 50 mM stock in DMA, with additional DMA pre-dosed to achieve final 5% v/v of DMA. The reaction was incubated for 3 h at 20° C. before it was quenched with the addition of 6 equivalents of glycine over antibody added from a 50 mM stock solution in water. The conjugate at DAR 4.0 was purified by G25 desalting followed by 6 volumes of buffer exchange using a 30 KDa ViVa membrane concentrator into 25 mM Histidine/Cl, pH 6.0, 200 mM sucrose. 2% w/v polysorbate 20 (PS20) was added to achieve final of 0.02% w/v of PS20 in 25 mM His/CI, pH 6.0, 200 mM sucrose.

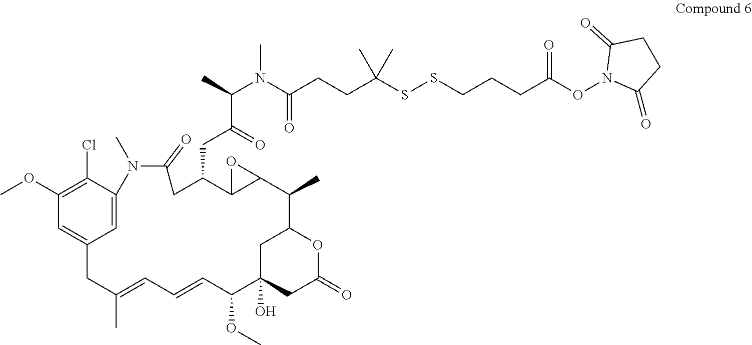 [SMILES: COc1cc2cc(c1Cl)N(C)C(=O)C[C@H](CC(=O)[C@@H](C)N(C)C(=O)CCC(C)(C)SSCCCC(=O)ON1C(=O)CCC1=O)C1OC1[C@@H](C)C1C[C@@](O)(CC(=O)O1)[C@H](OC)/C=C/C=C(\C)C2]

### Determination of Drug Antibody Ratio (DAR) for Cantuzumab Ravtansine by UV-VIS

The DAR value for Cantuzumab ravtansine was determined using UV-VIS analysis at 252 nm, 280 nm and 320 nm. Dilutions were performed in formulation buffer using a 1 cm path length quartz cuvette with blank correction with formulation buffer alone. The molar concentration of DM4 in the ADC sample was calculated following equation 1 with [DM4]=DM4 molar concentration, A280=absorbance at 280 nm-absorbance at 320 nm×dilution, A252=absorbance at 252 nm-absorbance at 320 nm×dilution, εDM4₂₅₂=26159 M⁻¹c⁻¹, εDM4₂₈₀=5180 M⁻¹c⁻¹.

$$[ DM 4 ] = \frac{A 252 - A 280 * \{ \frac{A 252}{A 280} \} Ab}{ε DM 4.252 - ε DM 4.28 * \{ \frac{A 252}{A 280} \} Ab} \\ Calculation for determining concentration ( mol / L ) of DM 4 \qquad Equation 1$$

The molar concentration of protein in the ADC sample was calculated following equation 2 with [Ab]=molar protein concentration, A280=absorbance at 280 nm-absorbance at 320 nm×dilution, εDM4₂₈₀=5180 M⁻¹c⁻¹, εAb₂₈₀=223400 M⁻¹c⁻¹, [DM4]=molar concentration of DM4 in ADC sample, from equation 1.

$$[ Ab ] = \frac{A 280 - ε DM 4.28 * [ DM 4 ]}{ε AB \text{.280}} \\ Calculation for determining protein concentration ( mol / L ) for \\ the ADC , subtracting DM 4 contribution \qquad Equation 2$$

To calculate the DAR by UV analysis, the molar concentration of DM4 in the ADC sample is divided by the molar protein concentration of the ADC, as in equation 3. [DM4]=molar concentration of DM4 in ADC sample, from equation 1 and [Ab]=molar protein concentration from equation 2. A DAR of 4.04 was calculated using equations 1 to 3 and the results shown in Table 3.

$$DAR = \frac{[ DM 4 ]}{[ Ab ]} \\ DAR calculation by UV analysis , \\ combining results from equations 1 and 2 \qquad Equation 3$$

| | | | | A280 − | A252 − |
|---|---|---|---|---|---|
| Replicate | A320 | A280 | A252 | A320 | A320 |
| 1 | 0.002 | 0.4270 | 0.3280 | 0.425 | 0.326 |
| 2 | 0.002 | 0.4180 | 0.3210 | 0.416 | 0.319 |
| 3 | 0.002 | 0.4187 | 0.3220 | 0.417 | 0.320 |
| Average | 0.002 | 0.4212 | 0.3236 | 0.419 | 0.321 |
| Dilution | 20 | | | | |
| factor | | | | | |
| [DM4] | 1.3247 − 04 | | | | |
| [Antibody] | 3.27333E−05 | | | | |
| DAR average | 4.04 | | | | |

Table 3 shows triplicate UV-vis readings of Cantuzumab ravtansine and summary of DAR value calculated based on equations 1, 2 and 3.

### Determination of Monomeric Content of Cantuzumab Ravtansine Comparator

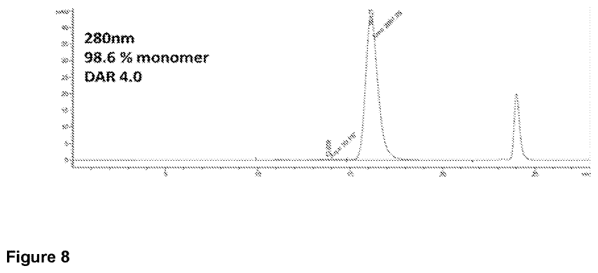

ADC was assessed for monomeric content and the presence of high molecular weight (HMW) aggregates, dimers, and fragments (LMW) using size exclusion chromatography (TOSOH TSKgel G3000SWXL 7.8 mm×30 cm, 5 μmcolumn). Running conditions: Flow at 0.5 mL/min in 10% IPA, 0.2 M Potassium phosphate, 0.25 M Potassium chloride, pH 6.95. Analysis of Cantuzumab ravtansine showed 98.6% monomeric ADC, as shown in FIG. 8.

### Example 6. Determination of CanAg Expression Levels on SNU-16, Colo205, BxPC3, HT29 and N87 Cell Lines Using Flow Cytometry

The expression of CanAg in five different cell lines was evaluated by comparison of a Phycoerythrin (PE)-conjugated HC1LC1 antibody to a Phycoerythrin-conjugated Isotype-matched control antibody (Biolegend, No: 403504), using a flow cytometry-based binding assay. Phycoerythrin-conjugated HC1LC1 antibody was prepared using the PE/R-Phycoerythrin Conjugation Kit-Lightning-Link® kit (Abcam, ab102918) following manufacture instructions. The five cell lines tested were SNU-16 (gastric cancer cell line), Colo-205 (human colorectal cancer cell line), BxPC-3 (human pancreatic epithelial adenocarcinoma cells), N87 (human gastric carcinoma) and HT29 (human colorectal adenocarcinoma cells). Cells were harvested and resuspended with FACS buffer and counted. 2×10⁵ cells were aliquoted and washed once with 3 ml of FACS buffer. Cells were then resuspended with 100 uL FACS buffer containing 2 μL (20 μg/ml) of HC1LC1 antibody-PE or the Isotype control-PE. Cells were incubated with PE-conjugates at 4° C. for 30 minutes followed by washing with 3 mL of FACS buffer twice. Cells were re-suspended in 500 μL FACS buffer for flow cytometry analysis. BD Quantibrite™ PE beads (BD Biosciences, 340495) were reconstituted with 0.5 mL 0.5% BSA and analysed following the manufacturer's instructions.

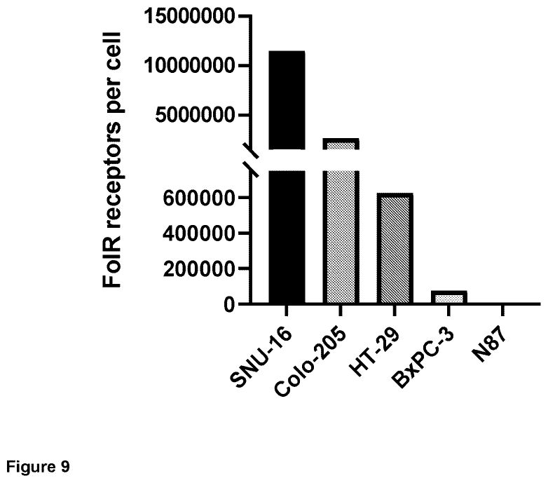

The PE geometric means of the different HC1LC1 antibody-PE concentrations were exported and the Log 10 values were calculated. Log 10 values were also calculated for the number of PE molecules per bead, based on lot-specific values, provided by the manufacturer. A linear regression of Log 10 values for PE geometric means against the number of PE molecules per bead was generated. To determine PE molecules per cell, Log 10 PE geometric means were substituted into the equation and the anti-Log determined. As shown in FIG. 9 and Table 4, SNU-16 and Colo205 cells were found to be the highest expressors of CanAg with an average of 11.5 million and 2.6 million PE molecules per cell. HT-29 showed moderate expression of CanAg with an average of 626,000 PE molecules per cell. BxPC-3 showed low expression of CanAg with an average of 75,000 PE molecules per cell. N87 cells showed very low expression of CanAg with an average of 2,500 PE molecules per cell.

| | HC1-LC1-PE |
|---|---|
| Cell Line | (20 μg/mL) |
| SNU-16 | 11487166 |
| Colo205 | 2613723 |
| HT-29 | 625956.4 |
| BxPC3 | 75335.7 |
| N87 | 2566.6 |

Table 4 shows calculations of PE molecules on SNU-16Colo205, BxPC3 and HT29 cell lines.

### Example 7. Determination of β-Glucuronidase Activity in Colo205, BxPC3, HT29 and N87 Cell Lines Using Fluorometric Assay

Activity of β-glucuronidase enzyme was determined in cell lines using β-glucuronidase Activity Assay Kit (Abcam, Ab234625). Cells were counted and 1×10⁷ cells were collected. Cells were washed once with 1 mL DPBS and centrifuged at 400×g for 5 minutes. Supernatant was discarded and cells were lysed with 500 μl assay buffer (Colo205, HT-29 and BxPC3 cells) or 300 μl assay buffer (N87 cells) and homogenized by ultrasonic cell disruptor. Lysate was centrifuged at 10,000×g for 5 minutes at 4° C. and supernatant was collected. 50 μL of supernatant was added to wells of a black 96-well plate. The volume was adjusted to 90 μL with β-Glucuronidase Assay Buffer. 5 μL of the reconstituted Positive control was mixed with B-Glucuronidase Assay Buffer to have 90 μL solution. 200 μM solution of 4-Methylumbelliferone (4-MU) standard was prepared in B-Glucuronidase Assay Buffer and 0, 0.5, 1, 2, 4, 6, 8, 10 μL of 200 μM 4-MU standard was added into a series of wells and the volume of each reaction was adjusted to 100 μL with B-Glucuronidase Assay Buffer to generate 0, 0.1, 0.2, 0.4, 0.8, 1.2, 1.6, and 2.0 nmol of 4-MU per well respectively. Substrate solution was 10-fold diluted in B-Glucuronidase Assay Buffer. 10 μL of the Substrate was added the Positive control and test samples. Fluorescence (Ex/Em=330/450 nm) was measured immediately after addition of substrate for 60 minutes at 37° C. recording every 2 min.

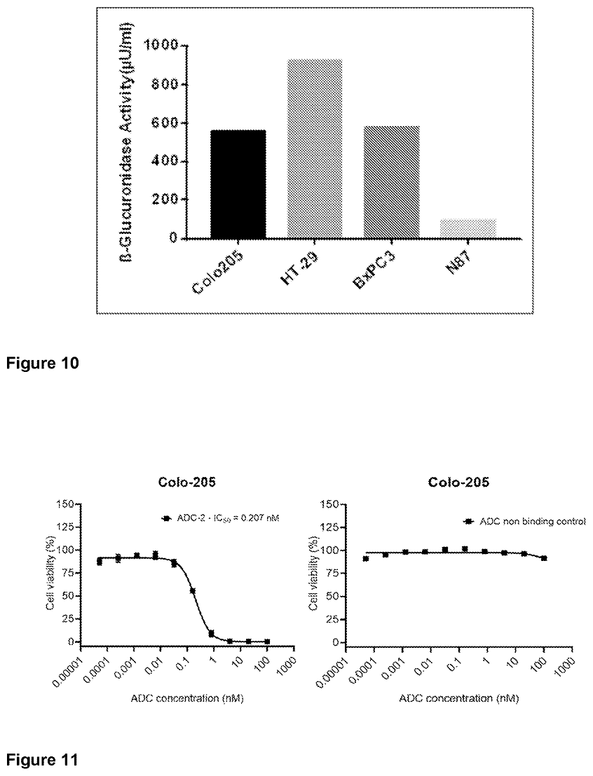

The results are shown in FIG. 10 with the β-glucuronidase level on 4 cell lines normalized to volume. HT29 cells were found to have the highest activity of β-glucuronidase enzyme. Colo-205 and BxPC3 cells were found to have similar activity of β-glucuronidase, and N87 cells showed the lowest activity of the enzyme.

### Example 8. In Vitro Activity Assays

In vitro activity was assessed using the luminescence-based Cell Titre-Glo (CTG) assay (Promega, No: G7572), which quantitates the amount of ATP present as a measure of viable cells. Specificity of cell killing was shown by incubating cells with a non-binding ADC control composed of the same payload.

Anti-CanAg ADC in vitro activity was evaluated in the colorectal cancer cell lines, Colo-205 (ATCC: CCL-222) and HT-29 (ATCC: HTB-38), the pancreatic adenocarcinoma cell line, BxPC-3 (ATCC: CRL-1687), and N87 gastric carcinoma (ATCC: CRL-5822). The ADC non-binding control was an anti-CD19 ADC (DAR 2.0) composed of the same linker-drug combination as ADC-3.

Cells were trypsinised and seeded to 96 well microplates in appropriate complete medium (Colo-205, BxPC-3 and N87-RPMI-1640 with 20% heat inactivated fetal bovine serum (FBS); HT-29-McCoy's-5A medium with 20% heat inactivated FBS) for 24 h at 37° C., 5% CO₂. Cells were seeded at density of 4000 (Colo-205) or 5000 (BxPC-3, HT-29, N87) cells per well, in a volume of 100 μL. After incubation, media was removed and replaced with 100 μL of fresh appropriate growth media. ADC-2 was 3-fold serially or 5-fold serially diluted in appropriate growth media to have a range of the following concentrations of ADC in 100 μL: BxPC-3 and HT-29-1000 nM, 333.3 nM, 111.1 nM, 37 nM, 12.34 nM, 4.1 nM, 1.37 nM, 0.45 nM, 0.15 nM, 0.051 nM; Colo-205-100 nM, 20 nM, 4 nM, 0.8 nM, 0.16 nM, 0.032 nM, 0.0064 nM, 0.0013 nM, 0.00026 nM, 0.000051 nM; N87-1000 nM, 200 nM, 40 nM, 8 nM, 1.6 nM, 0.32 nM, 0.06 nM, 0.013 nM, 0.0026 nM, 0.0013 nM.

For the comparison of ADC-1, ADC-2 and ADC-3, cells were trypsinised and seeded to 96 well microplates in appropriate complete medium (RPMI-1640 with 10% FBS) for 24 h at 37° C., 5% CO₂. Cells were seeded at a density of 4000 (Colo-205) cells per well, in a volume of 100 μL. After incubation, the media was removed and replaced with 100 μL of fresh appropriate growth media. ADCs were 5-fold serially diluted in appropriate growth media to have a range of the following concentrations of ADC in 100 μL: 50 nM, 10 nM, 2 nM, 0.40 nM, 0.08 nM, 0.016 nM, 0.0032 nM, 0.00064 nM 0.000128 nM, 0.0000256 nM, 0.0000051 nM, 0.000001 nM.

For the comparison of ADC-3 with Cantuzumab ravtansine and a non-binding ADC control cells were trypsinised and seeded to 96 well microplates in appropriate complete medium (RPMI-1640 with 10% FBS) for 24 h at 37° C., 5% CO₂. Cells were seeded at a density of 4000 (Colo-205) cells per well, in a volume of 100 μL. After incubation, the media was removed and replaced with 100 μL of fresh appropriate growth media. All ADCs were 3-fold serially diluted in appropriate growth media to have a range of the following concentrations of ADC in 100 μL: 50 nM, 16.67 nM, 5.56 nM, 1.85 nM, 0.62 nM, 0.206 nM, 0.069 nM, 0.023 nM, 0.0076 nM, 0.0025 nM, 0.00085 nM, 0.00028 nM. Cells were incubated with ADCs for 3 days (72 h). After ADC treatment, CTG reagent and cell plates were kept at RT for 30 minutes before CTG reagent addition. 100 μL of CTG reagent was added to each well and plates were shaken for 30s. Then the plate was incubated for 20 minutes at RT followed by luminescence measurements.

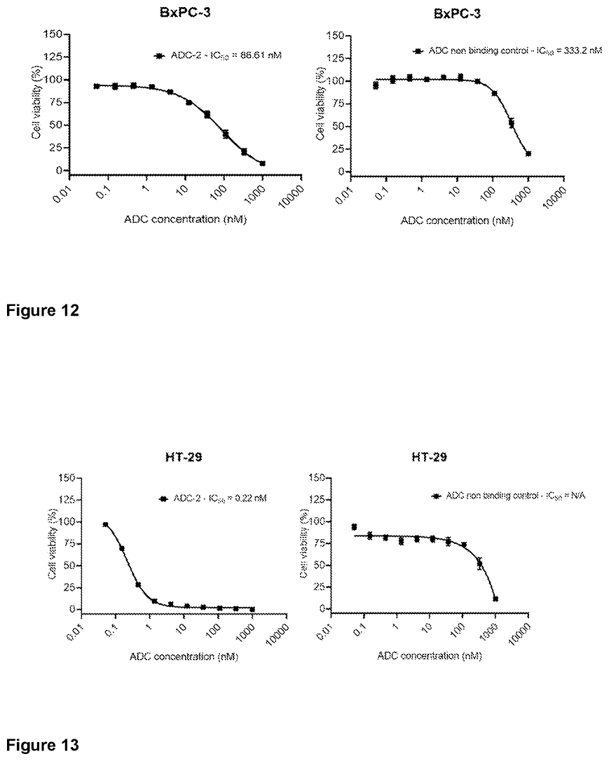

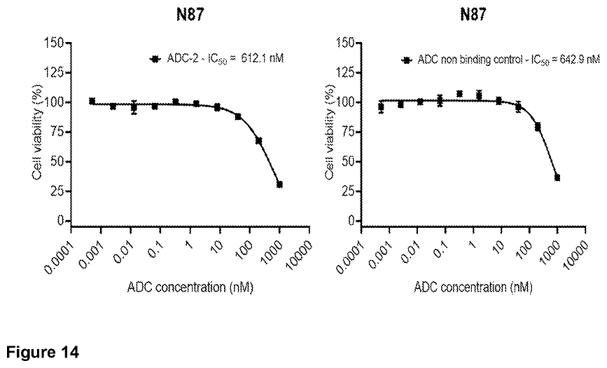

The results are shown in FIGS. 11 to 16. IC50 of ADC-2 correlates with CanAg expression level on Colo205, BxPC3 and N87 cells (see FIGS. 11, 12 and 14). Colo205 and BxPC3 cell lines have the same activity of β-glucuronidase (see FIG. 10). N87 cells showed the lowest amount of β-glucuronidase activity and CanAg expression level (see FIGS. 10 and 9), and ADC-2 showed very limited activity on N87 cells (see FIG. 14). The activity of ADC-2 on HT-29 was similar to the activity on Colo205 cells in spite of the lower expression level of CanAg on HT-29 (see FIG. 13) but the β-glucuronidase activity was the highest on HT-29 (see FIG. 10). This demonstrates that β-glucuronidase activity is also a factor contributing to the observed ADC activity. ADC-2 induced 100% cell killing on Colo-205, HT-29 and BcPC3. The incubation of cells with a non-binding ADC confirmed the specific activity of ADC-2 (see FIGS. 11 to 14).

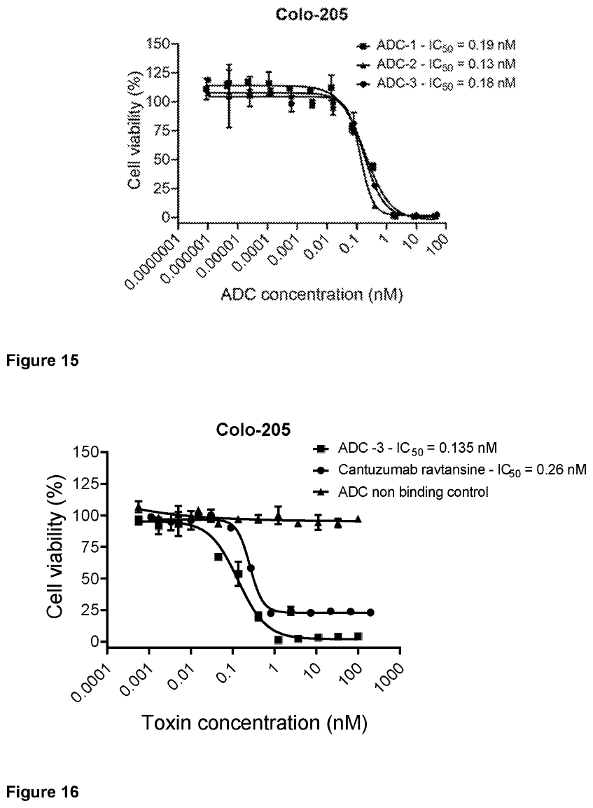

As shown in FIG. 15, ADC1, ADC-2 and ADC-3 all showed similar potent activity on Colo 205 cells, indicating that ADC in vitro activity is independent of the conjugation chemistry used in this cell line. Significantly, Cantazumab ravtansine is less efficient in killing Colo205 cancer cells as only 75% of cells were killed (see FIG. 16).

### Example 9. In Vivo Efficacy Studies in Colo-205 Xenograft Model

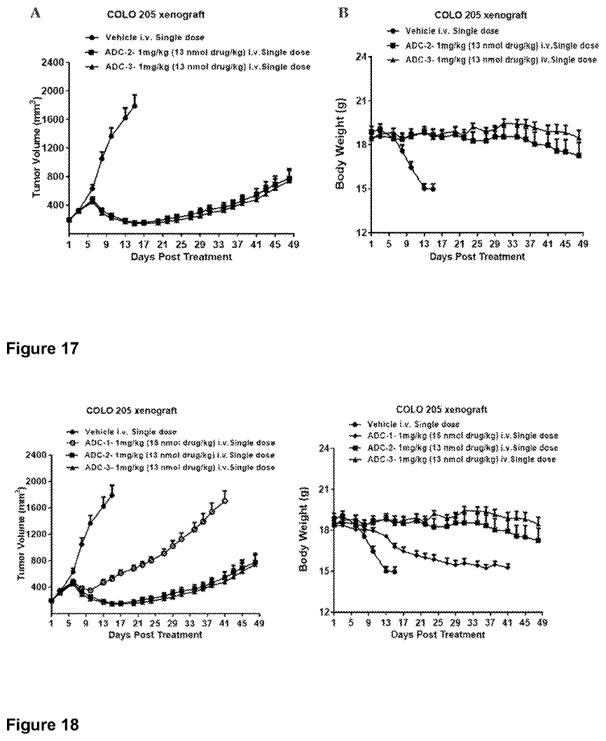

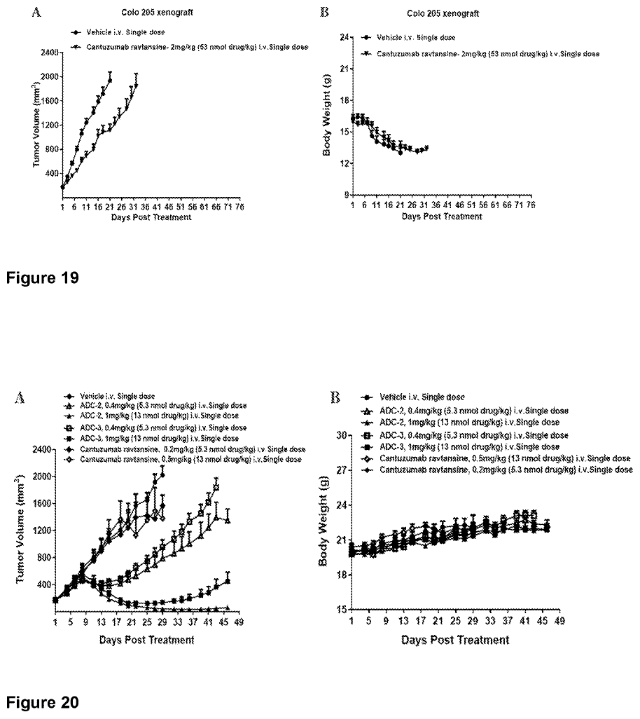

ADC1, ADC-2, ADC-3 and Cantuzumab ravtansine were evaluated in female CB17 SCID mice bearing Colo-205 xenograft. Mice were subcutaneously inoculated into the right flank with 5×10⁶ Colo-205 cells in 0.2 mL of DPBS mixed 1:1 with BD Matrigel. Tumour-bearing mice were randomized into groups of 5 animals each and treated with a single intravenous dose of ADC or alternatively with a vehicle solution (30 mM histidine, 200 mM sorbitol, 0.02% PS20 (w/v)) when the average tumour volume reached approximately 170 mm³ (Cantuzumab ravtansine) or 190 mm³ (ADC-1, ADC-2 and ADC-3). Conjugate doses of 1 mg/kg, for ADC-1, ADC-2 and ADC-3 (13 nmol drug/kg), and 2 mg/kg (53 nmol drug/kg) for Cantuzumab ravtansine were used for the Colo-205 xenograft study. Tumour size was measured thrice weekly in two dimensions using a calliper, and the volume was expressed in mm³ using the formula: V=0.5 a×b² where a and b are the long and short diameters of the tumour, respectively (see FIGS. 17A, 18 A and 19 A). The tumour size was then used for calculations of TGI (%) values (see Table 5). TGI, representing antitumor effectiveness, were calculated using the formula TGI (%)=[1-(Vₜᵣₑₐₜ₋ₜ−Vₜᵣₑₐₜ₋₁)/(V_{control-t}−V_{control-1})]×100, where Vₜᵣₑₐₜ₋₁ and V_{control-1} are the mean volumes of the treated and control groups on grouping day; Vₜᵣₑₐₜ₋ₜ and V_{control-t} are the mean volumes of the treated and control groups on a given day. Animals were euthanized when tumour volumes reached 2000 mm³. Body weight was also measured thrice weekly as a measure of compound toxicity (see FIGS. 17 B, 18 B and 19 B).

The in vivo effect of ADCs on Colo205 tumour xenograft is shown in FIGS. 17, 18 and 19. ADC-1, ADC-2 and ADC-3 induced substantial tumour growth inhibition (79%, 103% and 102% TGI, respectively) at 1 mg/kg (13 nmol of payload/kg (ADC-2 and ADC-3) or 15 nmol of payload/kg (ADC-1) with no observable toxicity, for example, see FIGS. 17 and 18 and Table 5. Cantuzumab ravtansine showed 40% tumour growth inhibition at 2 mg/kg (53 nmol of payload/kg) equivalent of 4 mg/kg of ADC-2 and ADC-3 as per drug load (see FIG. 19A and Table 5).

| | TGI [%] |
|---|---|
| ADC type | at 15 day |
| Cantuzumab ravtansine (2 mg/kg, 53 nmol drug/kg) | 40* |
| ADC-1 (1 mg/kg, 15 nmol/kg) | 79 |
| ADC-2 (1 mg/kg, 13 nmol drug/kg) | 103 |
| ADC-3 (1 mg/kg, 13 nmol drug/kg) | 102 |

Table 5 shows Tumour Growth Inhibition (TGI) of ADCs tested in Colo-205 xenograft model on day 15 of the study. * TGI calculation for Cantuzumab ravtansine was based on day 16.

### Example 10. In Vivo Efficacy Studies in BxPC3 Xenograft Model

ADC-2, ADC-3 and Cantuzumab ravtansine were evaluated in female CB17 SCID mice bearing BxPC3 xenograft. Mice were subcutaneously inoculated into the right flank with 5×10⁶ BxPC3 cells in 0.2 mL of DPBS containing 50% BD Matrigel. Tumour-bearing mice were randomized into groups of 5 animals each and treated with a single intravenous dose of ADC or alternatively with a vehicle solution comprising PBS pH 7.4 (see FIG. 20) or 30 mM histidine, 200 mM sorbitol, 0.02% PS20 (w/v) (see FIG. 21) when the average tumour volume reached approximately 150-180 mm³. Conjugate doses of 0.4 mg/kg (5.3 nmol of conjugated drug/kg), 1 mg/kg (13 nmol of conjugated drug/kg) for ADC-2 and ADC-3, and 0.2 mg/kg (5.3 nmol of conjugated drug/kg) and 0.5 mg/kg (13 nmol of conjugated drug/kg) for Cantuzumab ravtansine were used. Tumour size was measured thrice weekly in two dimensions using a calliper, and the volume was expressed in mm³ using the formula: V=0.5 a×b² where a and b are the long and short diameters of the tumour, respectively (see FIGS. 20A and 21 A). The tumour size was then used for calculations of TGI (%) values (see Table 6). TGI, representing antitumor effectiveness, were calculated using the formula TGI (%)=[1−(Vₜᵣₑₐₜ₋ₜ−Vₜᵣₑₐₜ₋₁)/(V_{control-t}V_{control-1})]×100, where Vₜᵣₑₐₜ₋₁ and V_{control-1} are the mean volumes of the treated and control groups on grouping day; Vₜᵣₑₐₜ₋ₜ and V_{control-t} are the mean volumes of the treated and control groups on a given day. Animals were euthanized when tumour volumes reached 2000 mm³. Body weight was also measured thrice weekly as a measure of compound toxicity (see FIGS. 20 B and 21 B).

The in vivo effect of ADCs on BxPC3 tumour xenograft is shown in FIGS. 20 to 21. ADC-2 induced tumour growth inhibition (66.7 and 107% TGI) at doses such as 0.4 mg/kg (5.3 nmol of conjugated drug/kg) and 1 mg/kg (13 nmol of conjugated drug/kg) (see FIG. 20A and Table 6). Treatment of BxPC3 xenograft with ADC-3 showed lower activity (57% and 101% TGI) over ADC-2 at 0.4 mg/kg (5.3 nmol of conjugated drug/kg) and 1 mg/kg (13 nmol of conjugated drug/kg) doses. The Cantuzumab ravtansine comparator, 0.2 mg/kg (5.3 nmol of conjugated drug/kg) and 0.5 mg/kg (13 nmol of conjugated drug/kg) showed a weak anti-tumour response in comparison to the ADC-2 and ADC-3 treatment at the same doses (see FIG. 20)

For the Cantuzumab ravtansine comparator, a dose of 2 mg/kg (53 nmol drug/kg) induced complete tumour inhibition but the anti-cancer activity was not maintained as tumour started to regrowth after 32 days (see FIG. 21 A).

Table 6 shows Tumour Growth Inhibition (TGI) of ADCs tested in BxPC3 xenograft model on day 29 or 32 of the study.

| | TGI [%] at 29 |
|---|---|
| ADC name | or 32* day |
| ADC-2 (0.4 mg/kg, 5.3 nmol/kg) | 66.7 |
| ADC-2 (1 mg/kg, 13 nmol/kg) | 107 |
| ADC-3 (0.4 mg/kg, 5.3 nmol drug/kg) | 57.6 |
| ADC-3 (1 mg/kg, 13 nmol drug/kg) | 101.8 |
| Cantuzumab ravtansine (0.2 mg/kg, 5.3 nmol drug/kg) | 24.3 |
| Cantuzumab ravtansine (0.5 mg/kg, 13 nmol drug/kg) | 34.6 |
| Cantuzumab ravtansine (2 mg/kg, 53 nmol drug/kg) | 104* |

### Example 11. In Vivo Efficacy Studies in NCI-N87 Xenograft Model

ADC-3, a ADC non-binding control, Cantuzumab ravtansine and Enhertu were evaluated in female CB17 SCID mice bearing NCI-N87 gastric cancer xenograft.

Enhertu is the brand name for Trastuzumab deruxtecan, which is an ADC consisting of the humanised anti-Her2 antibody trastuzumab (Herceptin) covalently linked to the topoisomerase I inhibitor deruxtecan (DAR 8.0). Enhertu has been approved by the U.S. Food and Drug Administration (FDA) for the treatment of breast cancer or gastric or gastroesophageal adenocarcinoma

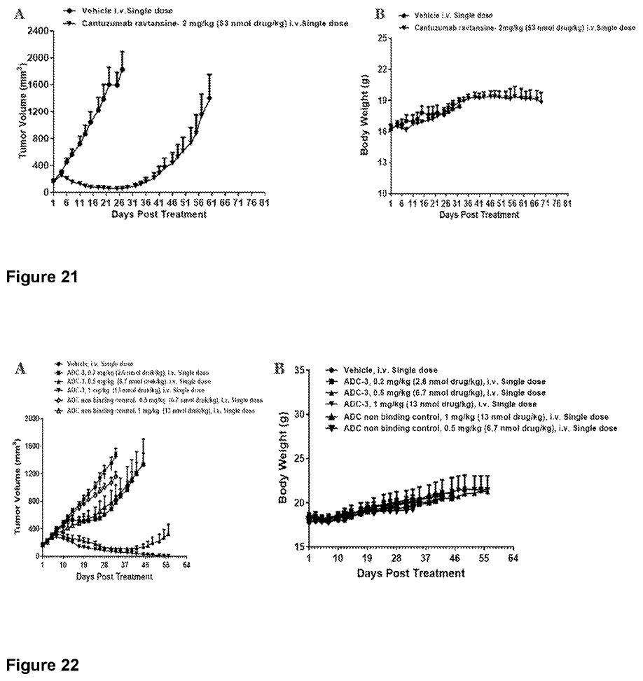

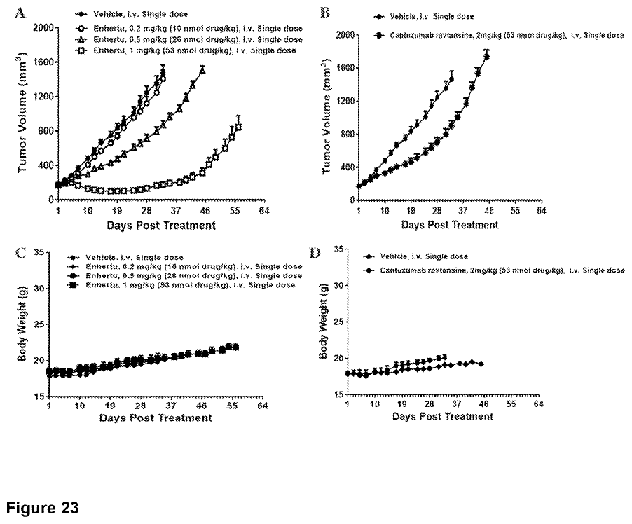

The ADC non-binding control was an anti-CD19 ADC (DAR 2.0) composed of the same linker-drug combination as ADC-3. Mice were subcutaneously inoculated into the right flank with 5×10⁶ NCI-N87 cells in 0.1 mL of DPBS containing 50% BD Matrigel. Tumour-bearing mice were randomized into groups of five animals each and treated with a single intravenous dose of ADC or alternatively with a vehicle solution (30 mM histidine, 200 mM sorbitol, 0.02% PS20 (w/v)) when the average tumour volume reached approximately 170-175 mm³. Conjugate doses of 0.2 mg/kg (2.6 nmol of conjugated drug/kg), 0.5 mg/kg (6.7 nmol of conjugated drug/kg) and 1 mg/kg (13 nmol of conjugated drug/kg) for ADC-3; 0.5 mg/kg (6.7 nmol of conjugated drug/kg) and 1 mg/kg (13 nmol of conjugated drug/kg) for ADC non-binding control; 0.2 mg/kg (10 nmol of conjugated drug/kg), 0.5 mg/kg (26 nmol of conjugated drug/kg) and 1 mg/kg (53 nmol of conjugated drug/kg) for Enhertu; 2 mg/kg (53 nmol of conjugated drug/kg) for Cantuzumab ravtansine were used. Tumour size was measured thrice weekly in two dimensions using a calliper, and the volume was expressed in mm³ using the formula: V=0.5 a×b² where a and b are the long and short diameters of the tumour, respectively (see FIGS. 22A, 23 A and B). The tumour size was then used for calculations of TGI (%) values (see Table 7). TGI, representing antitumor effectiveness, were calculated using the formula TGI (%)=[1−(Vₜᵣₑₐₜ₋ₜ−Vₜᵣₑₐₜ₋₁)/(V_{control-t}−V_{control-1})]×100, where Vₜᵣₑₐₜ₋₁ and V_{control-1} are the mean volumes of the treated and control groups on grouping day; Vₜᵣₑₐₜ₋ₜ and V_{control-t} are the mean volumes of the treated and control groups on a given day. Animals were euthanized when tumour volumes reached 2000 mm³. Body weight was also measured thrice weekly as a measure of compound toxicity (see FIGS. 22 B, 23 C and D).

The in vivo effect of ADCs on NCI-N87 tumour xenograft is shown in FIGS. 22 and 23. Interestingly, the in vitro analysis of ADC-2 on NCI-N87 cells described above showed no specific activity due to lack of CanAg expression on in vitro cell culture (see FIGS. 14 and 9). However, in this in vivo xenograft, ADC-3 showed target-specific activity in NCI-N87 as compared to the response of ADC non-binding control, which indicates the differences in CanAg expression on N87 cells cultured in vitro and in vivo xenograft.

ADC-3 induced 53% of specific tumour growth inhibition at a dose as low as 0.2 mg/kg (2.6 nmol of conjugated drug/kg) (see FIG. 22A and Table 7). In contrast, Cantuzumab ravtansine and Enhertu required higher doses (i.e 2 mg/kg (53 nmol of drug/kg) for Cantuzumab ravtansine; and 0.5 mg/kg (26 nmol drug/kg) for Enhertu) to achieve 43% and 46% tumour growth inhibition, respectively (see FIGS. 23A-B, Table 7). Further, although 1 mg/kg dose of Enhertu induced 99% TGI, tumour regrowth was observed after 28 days. On the other hand, ADC-3 dose of 0.5 mg/kg (6.7 nmol of conjugated drug/kg) and ADC-3 dose of 1 mg/kg (13 nmol drug/kg) induced 100% TGI by day 40 with no subsequent tumour recurrence for the 1 mg/kg dose (see FIG. 22A and Table 7).

| | TGI [%] |
|---|---|
| ADC name | at 33 day |
| ADC-3 (0.2 mg/kg, 2.6 nmol/kg) | 53 |
| ADC-3 (0.5 mg/kg, 6.7 nmol/kg) | 104 |
| ADC-3 (1 mg/kg, 13 nmol drug/kg) | 108 |
| Non-binding ADC control (0.5 mg/kg, 6.7 nmol/kg) | 24 |
| Non-binding ADC control (1 mg/kg, 13 nmol drug/kg) | 48 |
| Cantuzumab ravtansine (2 mg/kg, 53 nmol drug/kg) | 43 |
| Enhertu (0.2 mg/kg, 10 nmol/kg) | 4 |
| Enhertu (0.5 mg/kg, 26 nmol/kg) | 46 |
| Enhertu (1 mg/kg, 53 nmol/kg) | 99 |

Table 7 shows Tumour Growth Inhibition (TGI) of ADCs tested in NCI-N87 xenograft model on day 33 of the study.

Accordingly, in all cell lines tested, ADCs according to the present invention demonstrated superior TGI compared to the comparator anti-CanAg ADC, Cantuzumab ravtansine. Additionally, ADC-3 demonstrated comparable TGI in a gastric cancer xenograft at a near 8-fold lower conjugated drug concentration compared to Enhertu, which is an FDA approved ADC for use in the treatment of gastric cancer. Moreover, while Enhertu induced 99% TGI at higher conjugated drug concentrations (i.e. 1 mg/kg, 53 nmol/kg), tumour re-growth was observed after 28 days following administration, whereas no observable tumour re-growth was detected at day 55 following administration of ADC-3 at a dose of 1 mg/kg (13 nmol drug/kg). This result is even more surprising in view of the relative concentrations of conjugated drug used, i.e. 13 nmol/kg conjugated drug for ADC-3 versus 53 nmol/kg conjugated for Enhertu.

It will be appreciated by persons skilled in the art that the above embodiment has been described by way of example only and not in any limitative sense, and that various alterations and modifications are possible without departing from the scope of the invention as defined by the appended claims.

### Sequence Listings

In the following sequence listings, amino acids belonging to the signal peptide are shown in bold.

### 1. Murine Parental Sequence Used for Humanisation

-Murine c242 HC (CDR regions are shown underlined)
SEQ ID NO 1
MDWLRNLLFLMAAAQSIQAQVQLVQSGPELKKPGETVKISCKASDYTFTY
YGMNWVKQAPGKGLKWMGWIDTTTGEPTYAEDFKGRIAFSLETSASTAYL
QIKNLKNEDTATYFCARRGPYNWYFDVWGAGTTVTVSSAKTTPPSVYP
-Murine c242 LC (CDR regions are shown underlined)
SEQ ID NO 2
MRCLAEFLGLLVLWIPGAIGDIVMTQAAPSVPVTPGESVSISCRSSKSLL
HSNGNTYLYWFLQRPGQSPQLLIYRMSNLVSGVPDRFSGSGSGTAFTLRI
SRVEAEDVGVYYCLQHLEYPFTFGPGTKLELKRADAAPTVT

2. Murine c242 Sequence in hIgG1 (G1m17) Backbone

-pLEV123-Parental murine HC-hIgG1-murine Ab (Mu)
SEQ ID NO 3
MDPKGSLSWRILLFLSLAFELSYGQVQLVQSGPELKKPGETVKISCKASDYTFTYY
GMNWVKQAPGKGLKWMGWIDTTTGEPTYAEDFKGRIAFSLETSASTAYLQIKNLK
NEDTATYFCARRGPYNWYFDVWGAGTTVTVSSASTKGPSVFPLAPSSKSTSGGTA
ALGCLVKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKPKDTLMI
SRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQ
GNVFSCSVMHEALHNHYTQKSLSLSPG*
-pLEV123-Parental murine LC-hKappa
SEQ ID NO 4
METDTLLLWVLLLWVPGSTGDIVMTQAAPSVPVTPGESVSISCRSSKSLLHSNGNT
YLYWFLQRPGQSPQLLIYRMSNLVSGVPDRFSGSGSGTAFTLRISRVEAEDVGVYY
CLQHLEYPFTFGPGTKLELKRTVAAPSVFIFPPSDEQLKSGTASVVCLLNNFYPREA
KVQWKVDNALQSGNSQESVTEQDSKDSTYSLSSTLTLSKADYEKHKVYACEVTHQ
GLSSPVTKSFNRGEC*

### 3. Humanised Antibodies for CanAg Target

-pLEV123-HC1-hIgG1(G1m17)
SEQ ID NO 5
MDPKGSLSWRILLFLSLAFELSYGQVQLVQSGAEVKKPGASVKVSCKASDYTFTY
YGINWVRQAPGQGLEWMGWIDTTTGEPNYAQKLQGRVTFTLDTSASTAYMELRSL
RSDDTAVYYCARRGPYNWYFDVWGQGTLVTVSSASTKGPSVFPLAPSSKSTSGG
TAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLTVDKSRW
QQGNVFSCSVMHEALHNHYTQKSLSLSPG*
-pLEV123-HC2-hIgG1(G1m17)
SEQ ID NO 6
MDPKGSLSWRILLFLSLAFELSYGQVQLVQSGAEVKKPGASVKVSCKASDYTFTY
YGMNWVRQAPGQGLEWMGWIDTTTGEPSYAQKFQGRVTFTLDTSASTVYMELSS
LRSEDTAVYYCARRGPYNWYFDVWGQGTLVTVSSASTKGPSVFPLAPSSKSTSG
GTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSR
WQQGNVFSCSVMHEALHNHYTQKSLSLSPG*
-pLEV123-HC3-hIgG1(G1m17)
SEQ ID NO 7
MDPKGSLSWRILLFLSLAFELSYGQVQLVQSGAEVKKPGASVKVSCKASDYTFTY
YGINWVRQATGQGLEWMGWIDTTTGEPTYAQKFQGRVTFTLETSISTAYMELSSL
RSEDTAVYYCARRGPYNWYFDVWGAGTLVTVSSASTKGPSVFPLAPSSKSTSGGT
AALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQ
QGNVFSCSVMHEALHNHYTQKSLSLSPG*
-pLEV123-LC1-hKappa
SEQ ID NO 8
METDTLLLWVLLLWVPGSTGDIVMTQTPLSLPVTPGEPASISCRSSKSLLHSNGNT
YLYWYLQKPGQSPQLLIYRMSNRASGVPDRFSGSGSGTDFTLKISRVEAEDVGVY
YCLQHLEYPFTFGPGTKVDIKRTVAAPSVFIFPPSDEQLKSGTASVVCLLNNFYPRE
AKVQWKVDNALQSGNSQESVTEQDSKDSTYSLSSTLTLSKADYEKHKVYACEVTH
QGLSSPVTKSFNRGEC*
-pLEV123-LC2-hKappa
SEQ ID NO 9
METDTLLLWVLLLWVPGSTGDIVMTQSPLSLPVTPGEPASISCRSSKSLLHSNGNT
YLYWYLQKPGQSPQLLIYRMSNLASGVPDRFSGSGSGTDFTLKISRVEAEDVGVYY
CLQHLEYPFTFGPGTKVDIKRTVAAPSVFIFPPSDEQLKSGTASVVCLLNNFYPREA
KVQWKVDNALQSGNSQESVTEQDSKDSTYSLSSTLTLSKADYEKHKVYACEVTHQ
GLSSPVTKSFNRGEC*
-pLEV123-LC3-hKappa
SEQ ID NO 10
METDTLLLWVLLLWVPGSTGDIVMTQTPLSLSVTPGQPASISCKSSKSLLHSNGNT
YLYWYLQKPGQSPQLLIYRMSNLFSGVPDRFSGSGSGTDFTLKISRVEAEDVGVYY
CLQHLEYPFTFGPGTKVDIKRTVAAPSVFIFPPSDEQLKSGTASVVCLLNNFYPREA
KVQWKVDNALQSGNSQESVTEQDSKDSTYSLSSTLTLSKADYEKHKVYACEVTHQ
GLSSPVTKSFNRGEC*

### 4. HuC242 (Cantuzumab) Antibody

-H-GAMMA-1
SEQ ID NO 11
QVQLVQSGAEVKKPGETVKISCKASDYTFTYYGMNWVKQAPGQGLKWMGWIDTT
TGEPTYAQKFQGRIAFSLETSASTAYLQIKSLKSEDTATYFCARRGPYNWYFDVWG
QGTTVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLS
LSPGK
-L-KAPPA
SEQ ID NO 12
DIVMTQSPLSVPVTPGEPVSISCRSSKSLLHSNGNTYLYWFLQRPGQSPQLLIYRM
SNLVSGVPDRFSGSGSGTAFTLRISRVEAEDVGVYYCLQHLEYPFTFGPGTKLELK
RTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC

5. Humanised Antibodies with CAAX Tag (GGGGGGGCVIM) at LC C-Terminus

-pLEV123-HC1-hIgG1(G1m17)
SEQ ID NO 13
MDPKGSLSWRILLFLSLAFELSYGQVQLVQSGAEVKKPGASVKVSCKASDYTFTY
YGINWVRQAPGQGLEWMGWIDTTTGEPNYAQKLQGRVTFTLDTSASTAYMELRSL
RSDDTAVYYCARRGPYNWYFDVWGQGTLVTVSSASTKGPSVFPLAPSSKSTSGG
TAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLTVDKSRW
QQGNVFSCSVMHEALHNHYTQKSLSLSPG*
-pLEV123-HC2-hIgG1(G1m17)
SEQ ID NO 14
MDPKGSLSWRILLFLSLAFELSYGQVQLVQSGAEVKKPGASVKVSCKASDYTFTY
YGMNWVRQAPGQGLEWMGWIDTTTGEPSYAQKFQGRVTFTLDTSASTVYMELSS
LRSEDTAVYYCARRGPYNWYFDVWGQGTLVTVSSASTKGPSVFPLAPSSKSTSG
GTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSR
WQQGNVFSCSVMHEALHNHYTQKSLSLSPG*
-pLEV123-HC3-hIgG1(G1m17)
SEQ ID NO 15
MDPKGSLSWRILLFLSLAFELSYGQVQLVQSGAEVKKPGASVKVSCKASDYTFTY
YGINWVRQATGQGLEWMGWIDTTTGEPTYAQKFQGRVTFTLETSISTAYMELSSL
RSEDTAVYYCARRGPYNWYFDVWGAGTLVTVSSASTKGPSVFPLAPSSKSTSGGT
AALGCLVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQ
QGNVFSCSVMHEALHNHYTQKSLSLSPG*
-pLEV123-LC1-hKappa
SEQ ID NO 16
METDTLLLWVLLLWVPGSTGDIVMTQTPLSLPVTPGEPASISCRSSKSLLHSNGNT
YLYWYLQKPGQSPQLLIYRMSNRASGVPDRFSGSGSGTDFTLKISRVEAEDVGVY
YCLQHLEYPFTFGPGTKVDIKRTVAAPSVFIFPPSDEQLKSGTASVVCLLNNFYPRE
AKVQWKVDNALQSGNSQESVTEQDSKDSTYSLSSTLTLSKADYEKHKVYACEVTH
QGLSSPVTKSFNRGECGGGGGGGCVIM
-pLEV123-LC2-hKappa
SEQ ID NO 17
METDTLLLWVLLLWVPGSTGDIVMTQSPLSLPVTPGEPASISCRSSKSLLHSNGNT
YLYWYLQKPGQSPQLLIYRMSNLASGVPDRFSGSGSGTDFTLKISRVEAEDVGVYY
CLQHLEYPFTFGPGTKVDIKRTVAAPSVFIFPPSDEQLKSGTASVVCLLNNFYPREA
KVQWKVDNALQSGNSQESVTEQDSKDSTYSLSSTLTLSKADYEKHKVYACEVTHQ
GLSSPVTKSFNRGECGGGGGGGCVIM
-pLEV123-LC3-hKappa
SEQ ID NO 18
METDTLLLWVLLLWVPGSTGDIVMTQTPLSLSVTPGQPASISCKSSKSLLHSNGNT
YLYWYLQKPGQSPQLLIYRMSNLFSGVPDRFSGSGSGTDFTLKISRVEAEDVGVYY
CLQHLEYPFTFGPGTKVDIKRTVAAPSVFIFPPSDEQLKSGTASVVCLLNNFYPREA
KVQWKVDNALQSGNSQESVTEQDSKDSTYSLSSTLTLSKADYEKHKVYACEVTHQ
GLSSPVTKSFNRGECGGGGGGGCVIM

1. An antibody conjugate represented by Formula I or a pharmaceutically acceptable salt or solvate thereof:
Ab-(L-D)n Formula I:
wherein:
Ab is a humanised C242 antibody or antigen-binding fragment thereof;
D is a pyrrolobenzodiazepine dimer prodrug;
L is a linker connecting Ab to D;
n is an integer from 1 to 20;
Wherein:
D is represented by Formula (II): 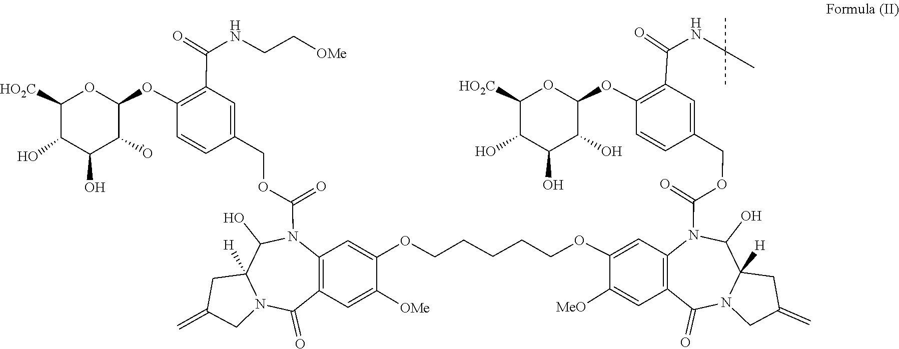
the linker comprises a central portion represented by Formula III, IV, V or isomers thereof: 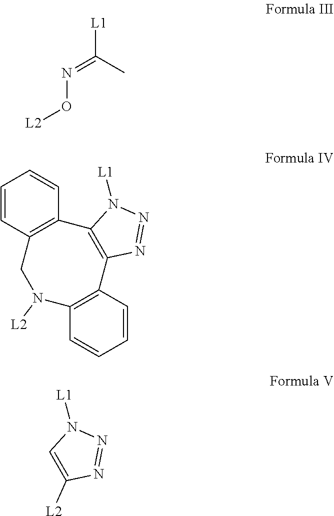
wherein:
L1 comprises a first connecting portion connecting the central portion to Ab; and
L2 comprises a second connecting portion connecting the central portion to D.

2. The antibody conjugate of claim 1, wherein L is covalently bound to Ab by a thioether bond, and optionally wherein the thioether bond comprises a sulfur atom of a cysteine of the Ab.

3. The antibody conjugate of claim 1, wherein the first connecting portion includes at least one isoprenyl unit represented by Formula VIII:

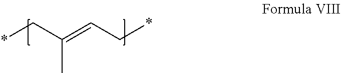

4. The antibody conjugate of claim 1, wherein the first connecting portion is represented by Formula IX: 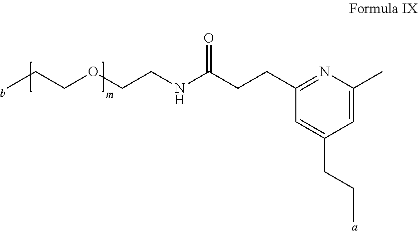
Wherein:
^{a} denotes a point of attachment to Ab;
^{b} denotes a point of attachment to the central portion; and
m is an integer from 1 to 20.

5. The antibody conjugate according to claim 1, wherein the second connecting portion comprises at least one polyethylene glycol unit represented by Formula X:
—(CH₂CH₂O)ₒ Formula X
wherein o is an integer from 1 to 10.

6. The antibody conjugate according to claim 1, wherein the second connecting portion is represented by Formula XI:
—(CH₂)ₚ—NHC(O)—(CH₂CH₂)O)_{q}(CH₂)ᵣ—C(O)—NH—(CH₂)ₛ—C(O) Formula XI
wherein:
p is an integer from 1 to 10;
q is an integer from 0 to 20;
r is an integer from 1 to 10; and
s is an integer from 1 to 10.

7. The antibody conjugate according to claim 1, wherein Ab includes one or more amino acid motifs that can be recognised by an isoprenoid transferase.

8. The antibody conjugate according to claim 7, wherein the isoprenoid transferase is FTase (farnesyl protein transferase) or GGTase (geranylgeranyl transferase).

9. The antibody conjugate according to claim 7, wherein the amino acid motif is CYYX, XXCC, XCXC, CXC or CXX, wherein C denotes cysteine, Y denotes an aliphatic amino acid, and X denotes an amino acid that determines substrate specificity of isoprenoid transferase.

10. The antibody conjugate according to claim 1, comprising a structure selected from: 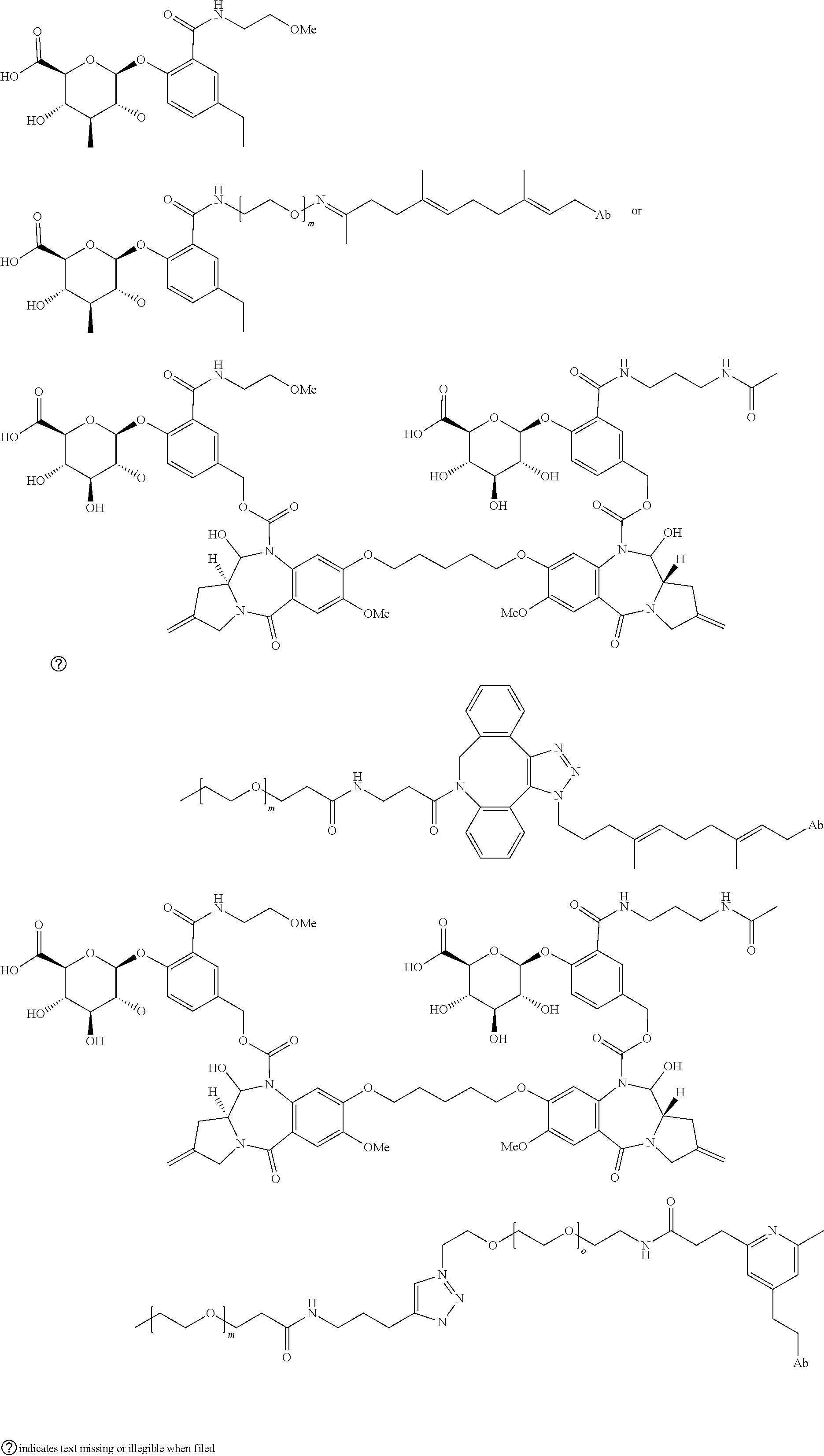
Wherein:
m is an integer from 0 to 20; and
o is an integer from 0 to 10.

11. A pharmaceutical composition comprising an antibody conjugate according to claim 1; and
one or more pharmaceutically acceptable excipients, diluents, or carriers.

12. The pharmaceutical composition according to claim 11 for use as a medicament.

13. The pharmaceutical composition according to claim 12, wherein the medicament is for use in the treatment of cancer.

14. The pharmaceutical composition according to claim 13, wherein the cancer is selected from the group consisting of lung cancer, small cell lung cancer, gastrointestinal cancer, colorectal cancer, bladder cancer, pancreatic cancer, biliary cancer, cervical cancer and uterine cancer.

15. The pharmaceutical composition according to claim 14, wherein the cancer is pancreatic cancer.

16. A method of treating cancer in a subject in need thereof, comprising the step of administering a therapeutically effective amount of the pharmaceutical composition according to claim 11 to the subject.

17. The method of claim 16, wherein the cancer is selected from the group consisting of lung cancer, small cell lung cancer, gastrointestinal cancer, colorectal cancer, bladder cancer, pancreatic cancer, biliary cancer, cervical cancer, and uterine cancer.

18. The method of claim 17, wherein the cancer is pancreatic cancer.


SEQUENCE LISTING (18 sequences)

SEQ ID NO: 1 - protein, 148 aa, Mus musculus
  SIGNAL 1..19
  BINDING 50..54: note = Complementarity-determining region
  BINDING 69..85: note = Complementarity-determining region
  BINDING 118..127: note = Complementarity-determining region
MDWLRNLLFLMAAAQSIQAQVQLVQSGPELKKPGETVKISCKASDYTFTYYGMNWVKQAPGKGLKWMGWIDTTTGEPTYAEDFKGRIAFSLETSASTAYLQIKNLKNEDTATYFCARRGPYNWYFDVWGAGTTVTVSSAKTTPPSVYP

SEQ ID NO: 2 - protein, 141 aa, Mus musculus
  SIGNAL 1..20
  BINDING 44..59: note = Complementarity-determining region
  BINDING 75..81: note = Complementarity-determining region
  BINDING 114..127: note = Complementarity-determining region
MRCLAEFLGLLVLWIPGAIGDIVMTQAAPSVPVTPGESVSISCRSSKSLLHSNGNTYLYWFLQRPGQSPQLLIYRMSNLVSGVPDRFSGSGSGTAFTLRISRVEAEDVGVYYCLQHLEYPFTFGPGTKLELKRADAAPTVT

SEQ ID NO: 3 - protein, 472 aa, Mus musculus
  SIGNAL 1..24
MDPKGSLSWRILLFLSLAFELSYGQVQLVQSGPELKKPGETVKISCKASDYTFTYYGMNWVKQAPGKGLKWMGWIDTTTGEPTYAEDFKGRIAFSLETSASTAYLQIKNLKNEDTATYFCARRGPYNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 239 aa, Mus musculus
  SIGNAL 1..20
METDTLLLWVLLLWVPGSTGDIVMTQAAPSVPVTPGESVSISCRSSKSLLHSNGNTYLYWFLQRPGQSPQLLIYRMSNLVSGVPDRFSGSGSGTAFTLRISRVEAEDVGVYYCLQHLEYPFTFGPGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 472 aa, synthetic construct
  SIGNAL 1..24
MDPKGSLSWRILLFLSLAFELSYGQVQLVQSGAEVKKPGASVKVSCKASDYTFTYYGINWVRQAPGQGLEWMGWIDTTTGEPNYAQKLQGRVTFTLDTSASTAYMELRSLRSDDTAVYYCARRGPYNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 472 aa, synthetic construct
  SIGNAL 1..24
MDPKGSLSWRILLFLSLAFELSYGQVQLVQSGAEVKKPGASVKVSCKASDYTFTYYGMNWVRQAPGQGLEWMGWIDTTTGEPSYAQKFQGRVTFTLDTSASTVYMELSSLRSEDTAVYYCARRGPYNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 472 aa, synthetic construct
  SIGNAL 1..24
MDPKGSLSWRILLFLSLAFELSYGQVQLVQSGAEVKKPGASVKVSCKASDYTFTYYGINWVRQATGQGLEWMGWIDTTTGEPTYAQKFQGRVTFTLETSISTAYMELSSLRSEDTAVYYCARRGPYNWYFDVWGAGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 239 aa, synthetic construct
  SIGNAL 1..20
METDTLLLWVLLLWVPGSTGDIVMTQTPLSLPVTPGEPASISCRSSKSLLHSNGNTYLYWYLQKPGQSPQLLIYRMSNRASGVPDRFSGSGSGTDFTLKISRVEAEDVGVYYCLQHLEYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 239 aa, synthetic construct
  SIGNAL 1..20
METDTLLLWVLLLWVPGSTGDIVMTQSPLSLPVTPGEPASISCRSSKSLLHSNGNTYLYWYLQKPGQSPQLLIYRMSNLASGVPDRFSGSGSGTDFTLKISRVEAEDVGVYYCLQHLEYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 239 aa, unidentified
  SIGNAL 1..20
METDTLLLWVLLLWVPGSTGDIVMTQTPLSLSVTPGQPASISCKSSKSLLHSNGNTYLYWYLQKPGQSPQLLIYRMSNLFSGVPDRFSGSGSGTDFTLKISRVEAEDVGVYYCLQHLEYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 449 aa, synthetic construct
QVQLVQSGAEVKKPGETVKISCKASDYTFTYYGMNWVKQAPGQGLKWMGWIDTTTGEPTYAQKFQGRIAFSLETSASTAYLQIKSLKSEDTATYFCARRGPYNWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 219 aa, synthetic construct
DIVMTQSPLSVPVTPGEPVSISCRSSKSLLHSNGNTYLYWFLQRPGQSPQLLIYRMSNLVSGVPDRFSGSGSGTAFTLRISRVEAEDVGVYYCLQHLEYPFTFGPGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 472 aa, synthetic construct; identical to SEQ ID NO: 5
  SIGNAL 1..24

SEQ ID NO: 14 - protein, 472 aa, synthetic construct; identical to SEQ ID NO: 6
  SIGNAL 1..24

SEQ ID NO: 15 - protein, 472 aa, synthetic construct; identical to SEQ ID NO: 7
  SIGNAL 1..24

SEQ ID NO: 16 - protein, 250 aa, synthetic construct
  SIGNAL 1..20
  BINDING 239..250: note = CAAX tag
METDTLLLWVLLLWVPGSTGDIVMTQTPLSLPVTPGEPASISCRSSKSLLHSNGNTYLYWYLQKPGQSPQLLIYRMSNRASGVPDRFSGSGSGTDFTLKISRVEAEDVGVYYCLQHLEYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGGGGCVIM

SEQ ID NO: 17 - protein, 250 aa, synthetic construct
  SIGNAL 1..20
  BINDING 240..250: note = CAAX tag
METDTLLLWVLLLWVPGSTGDIVMTQSPLSLPVTPGEPASISCRSSKSLLHSNGNTYLYWYLQKPGQSPQLLIYRMSNLASGVPDRFSGSGSGTDFTLKISRVEAEDVGVYYCLQHLEYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGGGGCVIM

SEQ ID NO: 18 - protein, 250 aa, synthetic construct
  SIGNAL 1..20
  BINDING 240..250: note = CAAX tag
METDTLLLWVLLLWVPGSTGDIVMTQTPLSLSVTPGQPASISCKSSKSLLHSNGNTYLYWYLQKPGQSPQLLIYRMSNLFSGVPDRFSGSGSGTDFTLKISRVEAEDVGVYYCLQHLEYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGGGGCVIM